(19) United States
(12) Patent Application Publication
(10) Pub. No.: US 2026/0144443 A1
(43) Pub. Date: May 28, 2026

(54) DEVICES, SYSTEMS, AND METHODS RELATING SENSING ELASTICITY OF BIOLOGICAL TISSUES

(71) Applicant: VANDERBILT UNIVERSITY, Nashville, TN (US)

(72) Inventor: Xiaoguang DONG, Nashville, TN (US)

(21) Appl. No.: 19/398,606

(22) Filed: Nov. 24, 2025

Related U.S. Application Data
(60) Provisional application No. 63/723,903, filed on Nov. 22, 2024.

(51) Int. Cl. A61B 5/00; G16H 40/67

(52) U.S. Cl. CPC A61B 5/0053; A61B 5/4255; G16H 40/67; A61B 2560/0204; A61B 2560/0462; A61B 2562/0261; A61B 2562/12

(57) ABSTRACT

Described herein are devices, systems, and methods relating to monitoring the elasticity of soft biological tissues, for example, in the gastrointestinal tract. Described herein are embodiments of a wirelessly actuated palpation mechanism integrated into a swallowable capsule device, offering in situ tissue elasticity measurement with minimal invasiveness. Such approach can employ a magnetic soft cantilever beam actuated by external magnetic fields to gently press against soft tissue. Mechanical stress and strain can be monitored by an onboard magnetic sensor and a strain gauge, allowing for accurate assessment of tissue elasticity. Additionally, wireless modules utilizing Bluetooth Low Energy and powered by a battery can facilitate real-time communication. The device and system operates under external magnetic field control, which can move freely over soft tissues during examinations and palpate suspicious areas

### CROSS-REFERENCE TO RELATED APPLICATION

This application claims priority to, and the benefit of, U.S. Provisional Patent Application Ser. No. 63/723,903, entitled “DEVICES, SYSTEMS, AND METHODS RELATING SENSING ELASTICITY OF BIOLOGICAL TISSUES” and filed on Nov. 22, 2024, the entire contents of which are incorporated herein by reference as if set forth in its entirety.

### BACKGROUND

Remotely sensing the biomechanical properties of biological tissues deep inside the human body could enable minimally invasive, long-term, and continuous monitoring and diagnosis of diseases at early stages (Lin et al. Nat. Rev. Mater. 7, 850-869, 2022). Existing sensing methods using wearable sensors, implantable electrical devices and pure medical imaging all have their own drawbacks. First, flexible microdevices as wearable sensors have been developed to sense various physiological properties of soft tissues mostly on the human skin, which can only sense physiological signals in a short range limited to a few millimeters close to the epidermis, limiting their application for deep tissue sensing. Second, implantable electrical sensors need a surgery process for the implantation and frequently cause tissue inflammation. Third, medical imaging methods such as standalone or wearable ultrasonography (Wang. et al. Nat. Biomed. Eng. 5, 749-758, 2021) and Magnetic Resonance Elastography (Venkatesh et al. J. Magn. Reson. 37 (3), 544-555, 2013) have a large penetration depth but the sensing resolution is either insufficient for early detection or inappropriate for patients with obesity or implanted devices. Lastly, passive, or active vibration sensors based on resonators on the skin, such as magnetic actuators based on Lorenz forces [16], can sense the elasticity but have limited sensing range close to the epidermis, restricting their application for deep tissue sensing. So far, it is still challenging to sense advanced tissue elasticity at locations deep inside the organs with minimal invasion.

Monitoring the elasticity of soft biological tissues in the gastrointestinal (GI) tract with minimal invasion holds promise for early diagnosis of intestinal fibrosis, colorectal cancer, and other diseases featuring abnormal elasticity. However, existing methods of sensing tissue elasticity have drawbacks such as insufficient resolution for elastography, and discomfort or the requirement of risky anesthesia for flexible endoscopes or implantable devices. Accordingly, there is a need to address the aforementioned deficiencies and inadequacies.

### SUMMARY

Described herein are palpitation units, capsules, systems, and methods of use.

In embodiments, described herein are palpitation units. Palpitation units as described herein can comprise a double-layer cantilever beam comprising a strain gauge sensor and a back layer, wherein the double-layer cantilever beam is physically deformable along at least one axis; a magnet; and a palpitation probe. In embodiments of palpitation probes described herein, the back layer of the double-layer cantilever beam comprises polydimethylsiloxane (PDMS). In embodiments of palpitation probes described herein, the strain gauge sensor comprises laser-induced graphene (LIG). In embodiments of palpitation probes described herein, the magnet is a NdFeB (N45 grade) magnet. In embodiments of palpitation probes described herein, the palpitation probe comprises a cylindrical structure. In embodiments of palpitation probes described herein, the palpitation probe is three-dimensionally printed (3D printed). In embodiments of palpitation probes described herein, the palpitation probe is on a side of the magnet opposite the back layer. In embodiments of palpitation probes described herein, the magnet and the palpitation probe can be on a distal end of the palpitation unit along the deformation axis.

Also described herein are capsule devices. Capsule devices as described herein can comprise any palpitation unit as described herein, a power source, a magnetic sense, and a wireless communication transceiver. In embodiments of capsule devices described herein, the power source can be a battery or other energy-harvesting cell[s]. In embodiments of capsule devices described herein, the power source is a battery. In embodiments of capsule devices described herein, the magnet of the palpitation probe can be situated in between a first and a second magnet of the magnetic sensor. In embodiments of capsule devices described herein, the wireless communication transceiver comprises a Bluetooth Low Energy (BLE) System-on-a-Chip (SoC) or other low energy wireless transmission/reception means.

Also described herein are systems. Systems according to the present disclosure can comprise any capsule device according to the present disclosure, an external magnetic field generator, a mobile application in wireless communication with the capsule device, the external magnetic field generator, or both. In embodiments of systems according to the present disclosure, the external magnetic field generator can also comprise one or more magnets. In embodiments of systems according to the present disclosure, the magnetic field generator is configured to generate a magnetic field of about 10 to about 50 MT. Systems as described herein can further comprise a personal computing device comprising the mobile application.

Described are methods of using a system according to the present disclosure. In embodiments, described herein are methods of using a system, comprising administering a capsule of any one of claims 9 to 12 to a subject in need thereof; administering a magnetic field to the subject with a magnetic field generator; collecting measurements from the capsule in the presence of the administered magnetic field with a mobile application. In embodiments of methods according to the present disclosure, the subject in need thereof is a subject having or suspected of having Chrohn's disease or inflammatory bowel disease. In embodiments of methods according to the present disclosure, the capsule is administered orally to the subject. In embodiments of methods according to the present disclosure, the magnetic field is administered to a region of interest (ROI) of the subject. In embodiments of methods according to the present disclosure, the ROI is a muscosa-interfacing region of the subject. In embodiments of methods according to the present disclosure, the region of interest comprises a part of the gastrointestinal (GI) tract.

Also described are methods of making a deformable strain gauge sensor. In embodiments, methods of making a deformable strain gauge sensor can comprise inducing a layer of graphene on a planar substrate; casting the graphene layer with a liquid polymer; transferring the casted layer onto a cured back layer; and patterning the transferred layer. In embodiments of methods of making a deformable strain gauge sensor, the planar substrate comprises a polyimide. In embodiments of methods of making a deformable strain gauge sensor, inducing is with a carbon dioxide (CO₂) laser. In embodiments of methods of making a deformable strain gauge sensor, the planar substrate comprises a polyimide tape. In embodiments of methods of making a deformable strain gauge sensor, the liquid polymer comprises polydimethylsiloxane (PDMS). In embodiments of methods of making a deformable strain gauge sensor, the cured back layer comprises polydimethylsiloxane (PDMS). In embodiments of methods of making a deformable strain gauge sensor, the patterning is with an ultraviolet (UV) laser machine. In embodiments of methods of making a deformable strain gauge sensor, the patterning is facilitated by applying negative pressure simultaneously in the delamination process of a conductor on a printed circuit board (PCB). In embodiments of methods of making a deformable strain gauge sensor, the conductor is copper.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1F: Overall design of a capsule device for palpating soft biological tissues. FIG. 1A. Concept of sensing tissue elasticity by a capsule device for monitoring GI diseases such as intestine fibrosis inside the colon. FIG. 1B. Illustration of the palpation mechanism for sensing tissue elasticity and the components of the capsule device. The palpation strain and stress are monitored by a strain gauge sensor and a magnetic sensor, respectively. FIG. 1C. Illustration of the electrical resistance of the strain gauge sensor and the external magnetic field when palpating soft materials of different elasticities. FIG. 1D. The data flow chart of the system including the external magnetic field generator, the capsule device, and the user interface on a mobile phone or a computer. FIG. 1E. Optical images of the capsule device with several components. Component 1: A wireless communication board with a Bluetooth Low Energy (LE) System-on-a-Chip (SoC) and a magnetic sensor. Component 2: A flexible palpation unit with a magnetic actuator and strain gauge sensor. Component 3: A coin Alkaline battery (4.5V, 18 mAh). FIG. 1F. Optical images of the electronic components of the capsule device.

FIGS. 2A-2E: Design and fabrication of the palpation unit in the capsule device. FIG. 2A. Illustration of the components of the palpation unit and their dimensions. The palpation unit has a soft beam, a strain gauge made of LIG, a permanent magnet, and a 3D printed palpation probe. FIG. 2B. Illustration of the electrical resistance of a strain gauge sensor as a function of its curvature. L and t are the length and thickness of the palpation beam, respectively. ΔR=R−R₀, where R₀ is the electrical resistance of the senor when there is no deflection. ρ is the radius of curvature of the palpation beam. s∈[0, L] is the material coordinate of the sensor. FIG. 2C. Illustration of the step-by-step fabrication process of the double-layer cantilever beam with an integrated LIG strain gauge sensor and a back layer. The material of the back layer is Polydimethylsiloxane (PDMS) with a Young's modulus of 125 kPa. FIG. 2D. Optical images of LIG strain gauge in a top view. The dimensions of the LIG strain gauge are marked. FIG. 2E. Optical image of the strain gauge sensor with a LIG layer and a PDMS layer in a side view.

FIGS. 3A-3F: Characterization of sensing material elasticity by the palpation unit in the capsule device. FIG. 3A. Free-body diagram of a capsule device when palpating a soft material actuated by external magnetic fields. The magnetic field magnitude is gradually increased to allow the palpation probe to press the soft material. Magnetic gradient pulling forces are induced on the two NdFeB magnets mounted on the bottom cap of the capsule. FIG. 3B. Video snapshots of a capsule device palpating a soft material when applying external magnetic fields of different magnitudes. The soft material used is gel wax. FIG. 3C. The magnitude of the applied external magnetic field as a function of time. FIG. 3D. The electrical resistance of the strain gauge sensor as a function of time when applying the magnetic field shown in FIG. 3C. FIG. 3E. The displacement of the tip of the capsule cantilever beam as a function of time when applying the external magnetic field shown in FIG. 3C. FIG. 3F. The electrical resistance of the strain gauge as a function of the tip displacement of the capsule device cantilever beam. d₀ is a pre-deflection when there is no external magnetic field applied.

FIGS. 4A-4H: Sensing the material elasticity with the palpation unit in the capsule device. FIG. 4A. Optical images of the capsule device palpating two soft materials with different Young's modulus (5.3 kPa and 37.6 kPa). Scale bar, 3 mm. FIG. 4B. The recorded external magnetic field magnitude as a function of time. The black dashed line indicates the maximum magnetic field value. FIG. 4C. The recorded electrical resistance of the LIG strain gauge as a function of time. The dashed lines mark the maximum electrical resistance of the LIG strain gauge. FIG. 4D. External magnetic field as a function of the electrical resistance of the LIG strain gauge when the capsule device is palpating different soft materials. B₀ is the sensed magnetic field by the on-board magnetic sensor without applying any external magnetic field. FIG. 4E. (i) Experimental setup for palpation using a mechanical tester (Hysitron BioSoft, Bruker AG.). The motion speed is 5 μm/s. (ii) Zoomed-in image of an indentation probe on a synthetic material. (iii) Illustrate of the probe dimension. FIG. 4F. Example test on five synthetic materials of different elastic modulus. FIG. 4G. Ground truth measurement of the Young's modulus of the soft materials using a mechanical tester. The Young's modulus of the soft material is estimated using the Hertzian contact model (C. E. Wu, K. H. Lin, J. Y. Juang, Tribol Int 2016, 97, 71). FIG. 4H. The Young's modulus of different soft materials as a function of the slope angle in FIG. 4D. The mapping provides a calibrated model for material elasticity prediction. Error bar indicates the standard deviation of n=5 trials.

FIGS. 5A-5I: Demonstration and characterization of the capsule device anchoring motion and locomotion. FIG. 5A. Illustration of the capsule device (i) being pressed to the substrate by magnetic gradient pulling force for anchoring, and (ii) being actuated to roll on the substrate by magnetic torques. g indicates the gravity direction. B-C. Characterization of the (FIG. 5B) normal loading force and (FIG. 5C) the material deformation for the capsule device. The soft substrate used is gel wax. Capsule weight, 2.34 g. Error bar indicates standard deviation for n=5 trials. FIG. 5D. Video snapshots of a tethered capsule device rolling on a substate (gel wax) by applying a rotating external magnetic field in a side view and top view. The two wires are only for powering. Scale bars, 5 mm. FIG. 5E. The curvilinear motion trajectory of a capsule device when navigating on a phantom (gel wax) steered by external magnetic fields. Scale bar, 5 mm. FIG. 5F. Illustration of the helical skin of the capsule. FIG. 5G. Video frames of the helical crawling motion of an untethered capsule in narrow gaps. The two wires are only for powering. FIG. 5H. Sequential images of resisting peristaltic motions by a tethered capsule. FIG. 5I. Sequential optical images of an untethered capsule device navigating through a segment of colon phantom.

FIGS. 6A-6H: Demonstration of sensing material elasticity by palpation using the capsule device. FIG. 6A. Optical image of a capsule device with on-board sensor sending data to a cell phone. FIG. 6B. The received time-varying magnetic field data on a host computer. g indicates the gravity direction. FIG. 6C. The received time-varying strain gauge electrical resistance data on a host computer. FIG. 6D. Illustration and image of a capsule device palpating a phantom made of heterogeneous materials of different Young's modulus. Scale bar, 15 mm. FIG. 6E. The ratio of the sensed magnetic field and electrical resistance of the capsule device on a phantom with heterogeneous materials of different Young's modulus. FIG. 6F. The predicted Young's modulus of the soft materials palpated by the capsule device based on a calibrated model. FIG. 6G. Illustration and video frames of palpating porcine tissues with or without a stiff rod underneath by a fully untethered capsule device. Scale bars, 5 mm. FIG. 6H. The predicted Young's modulus of the porcine tissues shown in FIG. 6G. Error bar indicates standard deviation for n=5 trials.

FIG. 7 is another illustration of an embodiment of an aspect of the present disclosure.

FIG. 8 shows additional aspects of the present disclosure, in particular an embodiment of a capsule device in a relaxed 100a position and palpitation unit in a deflected position 100b with the probe 115 touching the biological tissue 117.

FIGS. 9A and 9B show additional aspects of an embodiment of a palpitation unit 200 according to the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of genetics, microbiology, biochemistry, molecular biology, cellular biology, tissue culture, therapeutic administrations and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any device, system, composition, kit, or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to device, system, composition, kit, or methods encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with the micro-organisms in nature.

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living or non-living entity comprised of a plurality of cells. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “preventing” means to stop or hinder a disease, disorder, or symptom of a disease or condition through some action.

The term “reducing” means to diminish in extent, amount, or degree.

The term “therapeutic agent” as used herein refers to a therapeutic substance selected from a group consisting of, but not limited to, analgesics, anesthetics, anti-inflammatory agents, antiasthma agents, antibiotics (including penicillins), anticoagulants, antihistamines, antitussives, antihypertensive agents, antimuscarinic agents, antimycobacterial agents, antioxidant agents, antipyretics, immunosuppressants, immunostimulants, antiviral agents, bacteriostatic agents, bronchodilators, buffering agents, contrast media, corticosteroids, cough suppressants (expectorants and mucolytics), diagnostic agents, diagnostic imaging agents, free radical scavenging agents, growth factors, haemostatics, immunological agents, lipid regulating agents, muscle relaxants, proteins, peptides and polypeptides, prostaglandins, radio-pharmaceuticals, time release binders, anti-allergic agents, stimulants and anoretics, steroids, sympathomimetics, vasodilators, and xanthines.

The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition.

### DISCUSSION

Described herein are devices, systems, and methods relating to a wirelessly actuated palpation mechanism integrated into a swallowable capsule device, offering tissue elasticity measurement with minimal invasiveness (for example, in situ). Approaches described herein employ a magnetic soft cantilever beam actuated by external magnetic fields to gently press against soft tissue. Mechanical stress and strain are monitored by an onboard magnetic sensor and a strain gauge, allowing for accurate assessment of tissue elasticity. Additionally, wireless modules utilizing Bluetooth Low Energy and powered by a battery facilitate real-time communication. The device operates under external magnetic field control, which can move freely over soft tissues during examinations and palpate suspicious areas. The elasticity sensing mechanism was validated and assessed on both phantom structures and ex vivo porcine colon tissues. Capsule devices described herein hold significant promise for assessing tissue physiological conditions and facilitating early disease diagnosis in hard-to-reach areas of the body.

Palpation-based sensing mechanisms and the implemented capsule devices described herein offer a minimally invasive and non-disruptive method for evaluating tissue elasticity, making it a promising technology for disease diagnosis, treatment assessment, and early detection, especially in conditions like intestine fibrosis and early-stage colorectal cancer.

With its innovative features including active steering, tissue palpation, wireless data transmission, and power supply, capsule devices described herein hold the potential to advance medical practices and improve patient outcomes in diagnosis and monitoring IBD, colorectal cancer, and other diseases.

A novel fundamental mechanism of palpating soft materials to sense deep tissue elasticity with minimal invasion using the physical interaction between the robot body and soft tissues was reported. The elasticity sensing function is enabled by a novel palpation unit fully controlled by external magnetic fields, such that the tissue properties are estimated by solving a parameter estimation problem based on an intrinsic robot-tissue interaction model. For the first time, sensing tissue elasticity properties with on-board sensors was demonstrated.

In addition, integrating a camera into palpation capsules described herein may further allow identifying suspicious tissue areas though it remains challenging to ensure the compact size. One alternative solution may be integrating a small magnet onto a commercial endoscope which could be further used to monitor palpation capsules described herein (see FIGS. S14A-S14B of Exhibit 1 of U.S. Provisional Patent Application Ser. No. 63/723,903 (not shown) for proof-of-concept) to facilitate tissue identification. In certain aspects, biopsy needles may also be added to assist with specimen collection when in the subject.

### I. Palpitation Unit

Described herein are palpitation units. Palpitation units of the present disclosure can comprise a strain gauge sensor comprising a magnet that is capable of physical deformation that can be used in devices according to the present disclosure, and other devices that could utilize the sensing.

In embodiments, a palpitation unit can comprise a double-layer cantilever beam comprising a strain gauge sensor and a back layer. The strain gauge sensor can be formed with laser-induced graphene (or patterned liquid metal polymer) on a thin layer of polymer (PDMS, for example, or other biocompatible materials such as polyurethane or silicone elastomer) and then placed on a polymer backing (PDMS, for example). In certain aspects the thickness of the polymer backing can be “tuned” and adjusted to adjust the sensitivity and deformation of the strain gauge sensor.

The double-layer cantilever beam can comprise a magnet, for example, a NdFeB (N45 grade) magnet. Other ferromagnetic materials such as samarium-cobalt can be utilized in magnets according to the present disclosure. In certain aspects, the double-layer cantilever beam can comprise a probe. The probe can be 3D-printed, for example, using a biocompatible and/or rigid resin. In certain aspects, the magnet and the palpitation probe are on a distal end of the palpitation unit along the deformation axis on an end opposite a holder.

The holder (also referred to herein as an “anchor”) can be comprised of a polymer and in a semicircular shape or other suitable shape or material to anchor the cantilever beam to a surface and allow for deformation along the axis that follows from the holder to the magnet probe situated on the opposite side of the beam.

The double-layer cantilever beam can be deformable along at least one axis, for example, along its length along an axis extending from a magnet to a holder on an opposite side.

Also described herein are strain gauge sensors and methods of making. In an embodiment, a method of making a deformable strain gauge sensor, comprises inducing a layer of graphene on a planar substrate; casting the graphene layer with a liquid polymer; transferring the casted layer onto a cured back layer; and patterning the transferred layer.

In certain aspects, the planar substrate can comprise a polyimide.

In certain aspects, the inducing can be with a carbon dioxide (CO₂) laser or fiber-based laser.

In certain aspects, the planar substrate can comprise a polyimide tape or Polyethylene terephthalate (PET).

In certain aspects, the liquid polymer can comprise polydimethylsiloxane (PDMS).

In certain aspects, the cured back layer can comprise polydimethylsiloxane (PDMS) or other biocompatible materials such as polyurethane.

In certain aspects, the patterning can be with an ultraviolet (UV) laser machine.

In certain aspects, the patterning can be facilitated by applying negative pressure simultaneously in the delamination process of a conductor on a printed circuit board (PCB).

In certain aspects, the conductor can be copper

### II. Capsule Devices

Described herein are capsule devices comprising palpitation units as described herein. Capsule units as described herein can further comprise a power source, for example a lithium ion battery or other energy harvesting cells. Capsule units can further comprise a magnetic sensor, for example, two magnets of opposite polarity creating a magnetic field through which the probe and magnet of the palpitation device can transverse. Capsule devices can further comprise a wireless communication transceiver, for example, a Bluetooth-low-energy (BLE) unit for transmitting and/or receiving information.

Capsule devices as described herein can comprise two halves, where the two halves are sealed at the joint to prevent fluids or other materials from entering the capsule when the capsule has been administered to a subject. In certain aspects, the subject may also be a non-living phantom or ex vivo tissue from a subject.

Also described herein are systems comprising capsule devices as described herein. Systems can further comprise an actuator capable of generating a magnetic field in order to facilitate movement of the capsule device through the tissue or tissue of a subject.

In certain aspects, capsule devices as described herein can further comprise a helical structure or other protrusion on the outer surface to assist with movement of the capsule in a subject.

### III. Systems

Described herein are systems. In certain aspects, systems can comprise palpitation units and capsule devices according to the present disclosure.

In certain aspects, systems can comprise an external magnetic field generator (for example, an electromagnet array or wearable permanent magnet actuated by motors) The magnetic field generator can comprise an actuator capable of generating a magnetic field that can help drive or otherwise assist with motion of the capsule device in a subject.

In certain aspects, systems can further comprise a mobile application in wireless communication with the capsule device, the external magnetic field generator, or both. In certain aspects, systems further comprise further comprising a personal computing device comprising the mobile application.

In certain aspects, the magnetic field generator is configured to generate a magnetic field of about 10 to about 50 MT.

### IV. Methods of Use and Treatment

Also described are kits and methods of use of capsule devices as described herein. Methods as described herein can comprise administering a capsule device as described herein to a subject, administering a magnetic field to the subject with a magnetic field generator; and collecting measurements from the capsule in the presence of the administered magnetic field with a mobile application.

In certain aspects, the subject in need thereof can be a subject having or suspected of having Chrohn's disease, inflammatory bowel disease, or colon cancer.

In certain aspects, the capsule device is administered orally or rectally to the subject, or during a surgical procedure.

In certain aspects, the magnetic field is administered to a region of interest (ROI) of the subject, for example, comprises a part of the gastrointestinal (GI) tract. In certain aspects, the ROI can be a muscosa-interfacing region of the subject, for example, the upper colon or small intestine.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### V. Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1: Mucosa-Interfacing Capsule for In Situ Sensing the Elasticity of Biological Tissues

### A. Introduction

Accurate sensing of physiological properties deep inside the human body is crucial for monitoring and understanding disease progression, as well as providing rapid feedback for close-loop therapy. Sensing tissue elasticity provides vital information in the GI tract for disease diagnosis as many diseases such as intestine fibrosis (S. Mascharak, J. L. Guo, M. Griffin, C. E. Berry, D. C. Wan, M. T. Longaker, Nature Reviews Bioengineering 2024 2:4 2024, 2, 305; L. A. Johnson, E. S. Rodansky, K. L. Sauder, J. C. Horowitz, J. D. Mih, D. J. Tschumperlin, P. D. Higgins, Inflamm Bowel Dis 2013, 19, 891.) in inflammatory bowel diseases (IBD) (J. Zhao, D. Liao, R. Wilkens, K. Krogh, H. Glerup, H. Gregersen, Acta Biomater 2021, 130, 332; S. D'Alessio, F. Ungaro, D. Noviello, S. Lovisa, L. Peyrin-Biroulet, S. Danese, Nature Reviews Gastroenterology & Hepatology 2021 19:3 2021, 19, 169.] and early-stage colorectal cancer (F. Baidoun, K. Elshiwy, Y. Elkeraie, Z. Merjaneh, G. Khoudari, M. T. Sarmini, M. Gad, M. Al-Husseini, A. Saad, Curr Drug Targets 2020, 22, 998.) are characterized as the increased elasticity of soft biological tissue (C. F. Guimarães, L. Gasperini, A. P. Marques, R. L. Reis, Nature Reviews Materials 2020 5:5 2020, 5, 351). The elasticity of biological tissue is influenced by the changes in the extracellular matrix, cellular structures, and interactions within the tumor microenvironment. For example, in intestinal fibrosis, the tissue elasticity is distinguishable from that of the normal tissue. The increased elasticity of the intestinal wall is due to the chronic inflammation triggering fibrotic changes in the intestinal tissue leading to excess collagen and other extracellular matrix components (S. D'Alessio, F. Ungaro, D. Noviello, S. Lovisa, L. Peyrin-Biroulet, S. Danese, Nature Reviews Gastroenterology & Hepatology 2021 19:3 2021, 19, 169). In addition, in colorectal cancers (CRC) (F. Baidoun, K. Elshiwy, Y. Elkeraie, Z. Merjaneh, G. Khoudari, M. T. Sarmini, M. Gad, M. Al-Husseini, A. Saad, Curr Drug Targets 2020, 22, 998.), tumor progression is associated with increased tissue elasticity (M. Jang, J. An, S. W. Oh, J. Y. Lim, J. Kim, J. K. Choi, J. H. Cheong, P. Kim, Nature Biomedical Engineering 2020 5:1 2020, 5, 114) as additional stress may be generated by tumor cell growth in a confined space (J. Bauer, M. A. B. Emon, J. J. Staudacher, A. L. Thomas, J. Zessner-Spitzenberg, G. Mancinelli, N. Krett, M. T. Saif, B. Jung, Scientific Reports 2020 10:1 2020, 10, 1).

Tissue elasticity has emerged as a promising biomarker, which is crucial to early detection of diseases and evaluate treatment efficacy, offering a comprehensive understanding of the dynamic changes occurring within the soft tissues (K. Nan, V. R. Feig, B. Ying, J. G. Howarth, Z. Kang, Y. Yang, G. Traverso, Nature Reviews Materials 2022 7:11 2022, 7, 908). Conventionally, accessing tissue elasticity relies on employing endoscope cameras or force sensors on tethered surgical tools (M. Beccani, C. Di Natali, C. E. Benjamin, C. S. Bell, N. E. Hall, P. Valdastri, Sens Actuators A Phys 2015, 223, 180; S. Mckinley, A. Garg, S. Sen, R. Kapadia, A. Murali, K. Nichols, S. Lim, S. Patil, P. Abbeel, A. M. Okamura, et al., IEEE International Conference on Automation Science and Engineering 2015, 2015-October 1151.) which are invasive and may not be suitable for prolonged measurements. In addition, non-invasive medical imaging methods, such as Ultrasound Elastography is useful, but they may only detect advanced fibrosis (I. Sack, Nature Reviews Physics 2022 5:1 2022, 5, 25.) due to relative low sensitivity. Magnetic Resonance Elastography (I. Sack, Nature Reviews Physics 2022 5:1 2022, 5, 25; F. Avila, B. Caron, G. Hossu, K. Ambarki, S. Kannengiesser, F. Odille, J. Felblinger, S. Danese, M. Choukour, V. Laurent, et al., Dig Dis Sci 2022, 67, 4518) may not be suitable for patients with obesity and implantable devices. Moreover, passive, or active vibration sensors based on resonators on the skin, such as magnetic actuators based on Lorenz forces (E. Song, Z. Xie, W. Bai, H. Luan, B. Ji, X. Ning, Y. Xia, J. M. Baek, Y. Lee, R. Avila, et al., Nature Biomedical Engineering 2021 5:7 2021, 5, 759), can sense the elasticity but have limited sensing range close to the epidermis outside of the body, restricting their application for deep tissue sensing. Lastly, flexible and implanted electronic sensors (X. Yu, H. Wang, X. Ning, R. Sun, H. Albadawi, M. Salomao, A. C. Silva, Y. Yu, L. Tian, A. Koh, et al., Nature Biomedical Engineering 2018 2:3 2018, 2, 165) have been shown sensing tissue elasticity inside the body, but it requires open surgery for implantation and may potentially cause inflammation. It is currently difficult to provide early-stage screening of the disease spots based on tissue elasticity with minimal invasion in the human GI tract.

Sensing the elasticity of GI tract tissues deep inside the human body with minimal invasion could enable on-demand, and continuous monitoring of various GI tract diseases at early stages (M. Lin, H. Hu, S. Zhou, S. Xu, Nature Reviews Materials 2022 7:11 2022, 7, 850). Devices actuated by external magnetic fields are particularly promising as magnetic fields can safely penetrate most biological tissues allowing minimally invasive or non-invasive medical operations such as drug delivery or sensing (B. J. Nelson, S. Gervasoni, P. W. Y. Chiu, L. Zhang, A. Zemmar, Proceedings of the IEEE 2022, 110, 1028). Previously reported magnetic microdevices (B. Xiao, Y. Xu, S. Edwards, L. Balakumar, X. Dong, Adv Funct Mater 2024, 34, 2307751; C. Wang, Y. Wu, X. Dong, M. Armacki, M. Sitti, Sci Adv 2023, 9, DOI 10.1126/sciadv.adg3988.) can sense the properties of biological tissues such as pH (C. Wang, Y. Wu, X. Dong, M. Armacki, M. Sitti, Sci Adv 2023, 9, DOI 10.1126/sciadv.adg3988), mucus viscosity (B. Xiao, Y. Xu, S. Edwards, L. Balakumar, X. Dong, Adv Funct Mater 2024, 34, 2307751). On a larger scale, technologies such as swallowable capsule endoscopes (N. Shamsudhin, V. I. Zverev, H. Keller, S. Pane, P. W. Egolf, B. J. Nelson, A. M. Tishin, Med Phys 2017, 44, e91; J. Min, Y. Yang, Z. Wu, W. Gao, Adv Ther (Weinh) 2020, 3, 1900125; G. Ciuti, R. Caliò, D. Camboni, L. Neri, F. Bianchi, A. Arezzo, A. Koulaouzidis, S. Schostek, D. Stoyanov, C. M. Oddo, et al., Journal of Micro-Bio Robotics 2016 11:1 2016, 11, 1.) and ingestible electronics offer the ability to monitor a range of physiological parameters in real-time as they navigate through the gastrointestinal tract. These include vital signs like heart rate (G. Traverso, V. Finomore, J. Mahoney, J. Kupec, R. Stansbury, D. Bacher, B. Pless, S. Schuetz, A. Hayward, R. Langer, et al., Device 2023, 1, 100125), analysis of microbiota composition (M. Mimee, P. Nadeau, A. Hayward, S. Carim, S. Flanagan, L. Jerger, J. Collins, S. McDonnell, R. Swartwout, R. J. Citorik, et al., Science (1979) 2018, 360, 915), detection of gases (J. M. Stine, K. L. Ruland, L. A. Beardslee, J. A. Levy, H. Abianeh, S. Botasini, P. J. Pasricha, R. Ghodssi, Adv Healthc Mater 2023, 2302897), and various other properties (C. Steiger, A. Abramson, P. Nadeau, A. P. Chandrakasan, R. Langer, G. Traverso, Nature Reviews Materials 2018 4:2 2018, 4, 83). Despite these advancements, current technologies still face challenges in accurately measuring tissue elasticity, which is crucial for assessing the mechanical properties of tissues in conditions such as gastrointestinal diseases and disorders. So far, it remains challenging to develop wireless miniature devices that can sense tissue elasticity spatiotemporally in situ.

Here a wirelessly actuated palpation mechanism and a capsule device integrated with such mechanism to sense the elasticity of soft biological tissues is presented. The sensing mechanism utilizes external magnetic fields to deflect a magnetic cantilever beam, gently deforming the soft materials and facilitating elasticity estimation. In addition to a palpation unit, the capsule device integrates an on-board battery and interfacing electronic module equipped with Bluetooth LE for wireless communication. Notably, the magnetic field employed ranges from 10 to 50 mT, ensuring safety for the human body. Leveraging a rolling locomotion, the capsule device can navigate on phantoms, porcine tissues, and potentially the GI tract within the human body. With its ability for on-demand sensing of soft tissue elasticity, this capsule device holds potential as a promising tool for the early detection of intestine fibrosis and other GI tract diseases characterized by tissue elasticity change.

### B. Concept and Working Principle of a Capsule Device for Sensing Tissue Elasticity

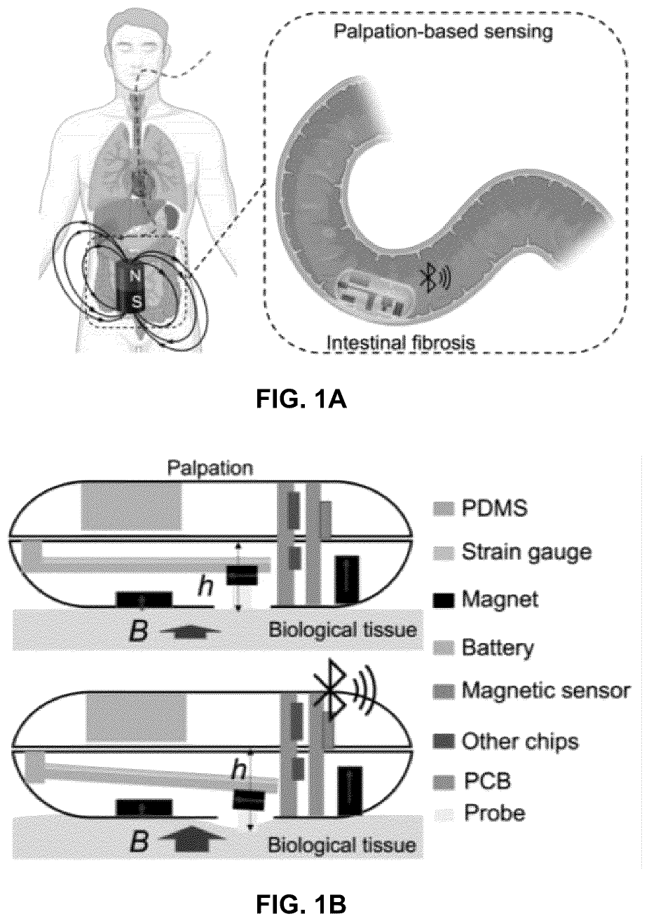

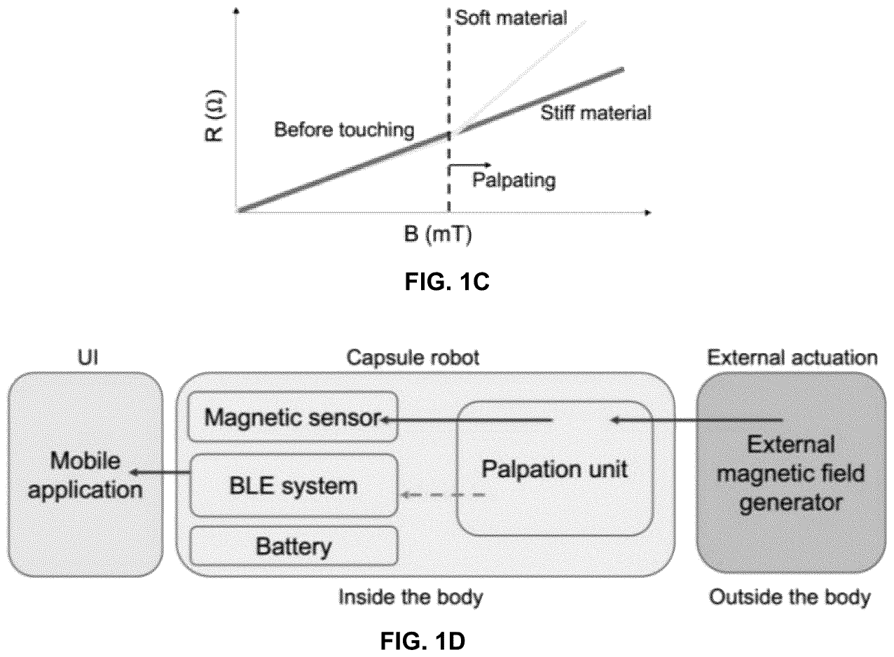

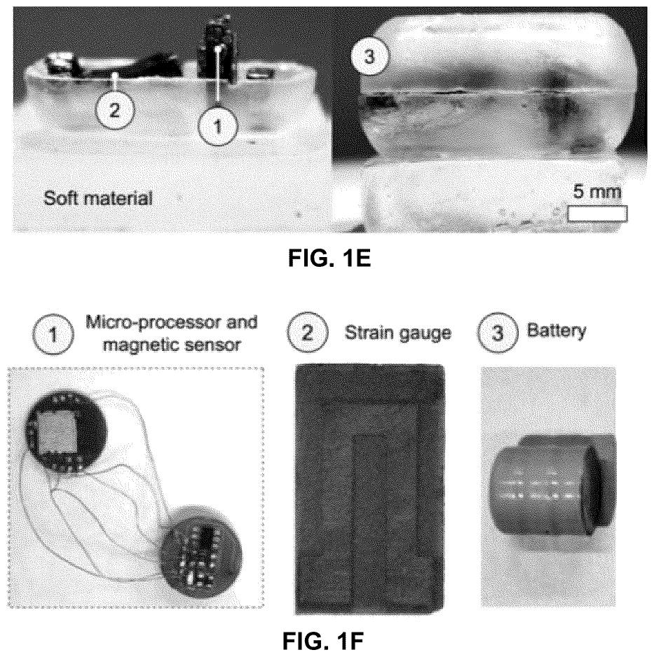

An overview of a capsule device with a palpation function remotely actuated and controlled via external magnetic fields is presented. The robot is designed for sensing within the GI tract, particularly in regions challenging to access using conventional endoscopes and colonoscopes, as shown in FIGS. 1A-1F. The capsule device has compact dimensions, measuring 15 mm in diameter and 32 mm in length (not shown, see, FIGS. S1A-S1B of Exhibit 1 of U.S. Provisional Patent Application Ser. No. 63/723,903), tailored for effective disease diagnosis in the GI tract, as showcased in FIG. 1A. FIG. 1B illustrates the integration of a palpation unit within the capsule device, enabling the sensing of local material elastic properties. This palpation unit features a cantilever beam with a magnetic tip, facilitating deflection upon the application of an external magnetic field. This deflection allows for the palpation of soft tissues with a probe, converting bending motion into compression motion. The stress applied correlates with the strength of the external magnetic field, a parameter accurately measured by an on-board magnetic field sensor. Simultaneously, a strain gauge sensor mounted on the cantilever beam precisely measures the deflection, forming the basis of an effective palpation mechanism for monitoring the elastic modulus of soft tissues. Please refer to “Fabrication of the electronic modules” in “Experimental Section” for the details of the electronic components.

FIG. 1C demonstrates how soft materials of different elasticities exhibit different deflection profiles when being palpated under identical magnetic field conditions. An approximate magnetic actuation field strength of 30 mT is deemed necessary for deforming the cantilever beam and generating sufficient palpation effects on soft materials, achievable using a permanent magnet positioned at a specified distance. Equipped with an integrated battery and wireless communication capabilities via Bluetooth LE, the capsule device can transmit real-time soft tissue information to mobile devices or cloud platforms for further analysis, as shown in FIG. 1D. In addition, FIG. 1E showcases an example of the capsule device equipped with both the palpation unit and the fully assembled electronic unit, with the individual components shown in FIG. 1F. For the schematics of the circuits design of these electronic components, please refer to FIGS. S2A and S2B of Exhibit 1 of U.S. Provisional Patent Application Ser. No. 63/723,903 (not shown) and FIGS. S3A and S3B of Exhibit 1 of U.S. Provisional Patent Application Ser. No. 63/723,903 (not shown). Lastly, it is emphasized that the on-board magnetic sensor can measure external magnetic fields without requiring knowledge of the exact distance from the sensor to the magnetic field generator. In practical scenarios, the capsule will be maneuvered to navigate soft tissues and locally palpate. Visualization of the surrounding tissue can be achieved by integrating a camera onboard or through external medical imaging methods such as ultrasound or X-ray imaging.

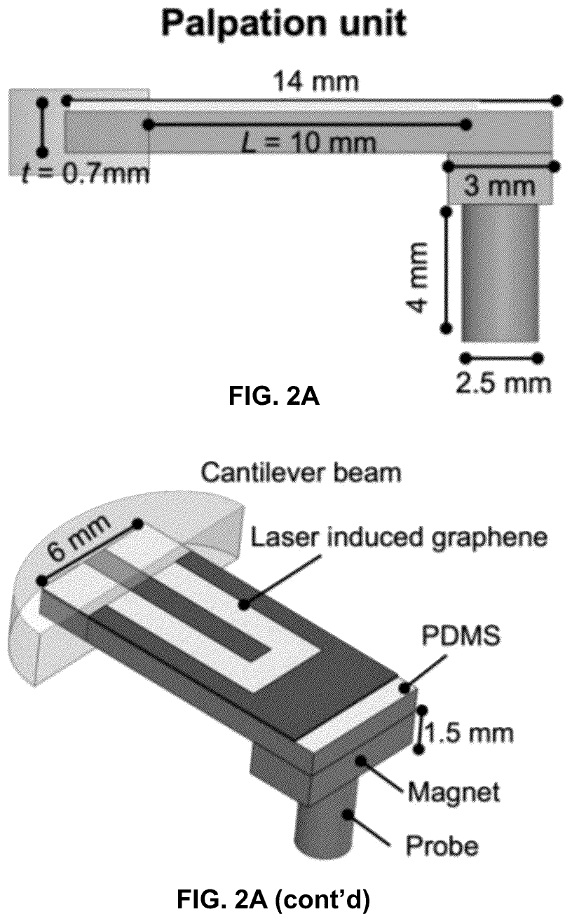

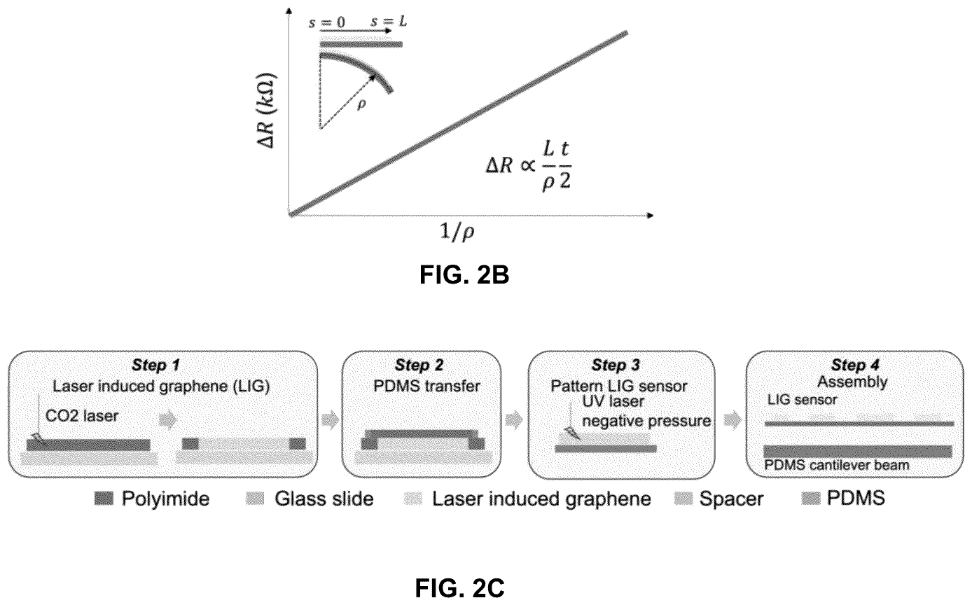

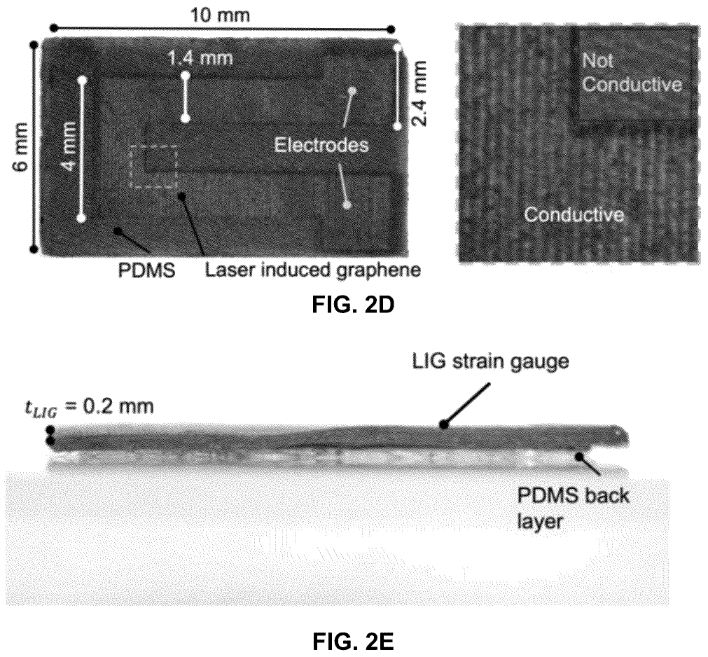

In FIGS. 2A-2E, the design and fabrication of an embodiment of the palpation unit is presented. As illustrated in FIG. 2A, this unit features a double-layer cantilever beam comprising a strain gauge sensor as the top layer and a pure PDMS layer as the bottom layer. At the tip of the cantilever beam, a NdFeB (N45 grade) magnet measuring 5 mm by 3 mm by 1.5 mm is securely affixed. Additionally, a 3D-printed probe of 2.5 mm in diameter and 4 mm in length is attached to the magnet, allowing transmitting the bending motion of the cantilever beam to the pushing motion of the probe. The strain gauge sensor serves a critical role in measuring the deflection of the cantilever beam, enabling accurate assessment of the deformation of soft materials. FIG. 2B presents the strain sensing mechanism utilizing the strain gauge sensor, wherein its electrical resistance R, which is proportional to the length of the sensor, exhibits an inverse relationship with ρ—the radius of curvature of the double-layer cantilever beam. This mechanism forms the foundation for precise deflection measurement within the palpation unit.

FIG. 2C outlines the fabrication process of the Laser-Induced Graphene (LIG) strain gauge, including several key steps. LIG-based strain gauge was used as it allows large deformation and is relatively easy to fabricate. Please refer to “Preparing the graphene layer” and “Patterning the LIG strain gauge sensor” in the “Experimental Section” for details. Briefly, the fabrication process includes inducing a thin layer of graphene onto a polyimide tape using a CO₂ laser, casting the graphene layer with liquid PDMS, and transferring it to cured PDMS after uniform heating. Subsequently, the transferred graphene layer atop the PDMS layer undergoes a patterning process using a UV laser machine (LPKF ProtoLaser U4), facilitated by applying negative pressure simultaneously in the delamination process of copper on a printed circuit board. FIGS. 2D and 2E provide optical images showcasing the strain gauge sensor with a specific pattern. These images underscore the strain gauge's resilience against a relatively large deformation and its relatively large gauge factor, ensuring effective measurement of the deflection angle within the palpation unit.

### Example 2: Sensing the Elasticity of Soft Materials by Palpation

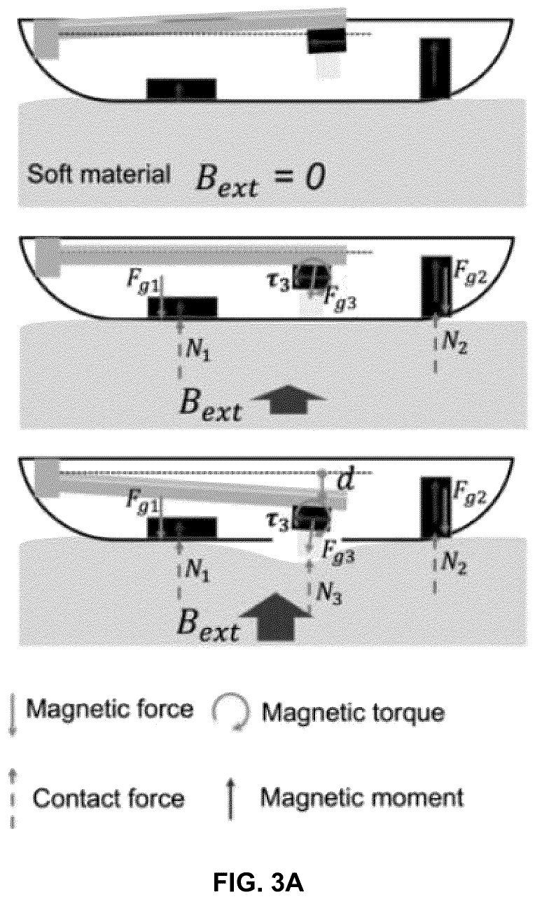

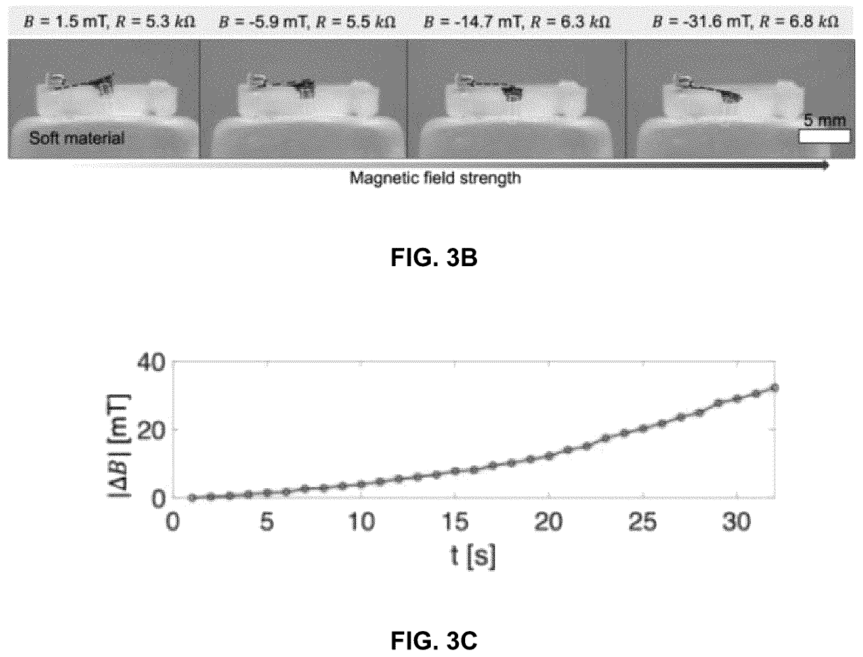

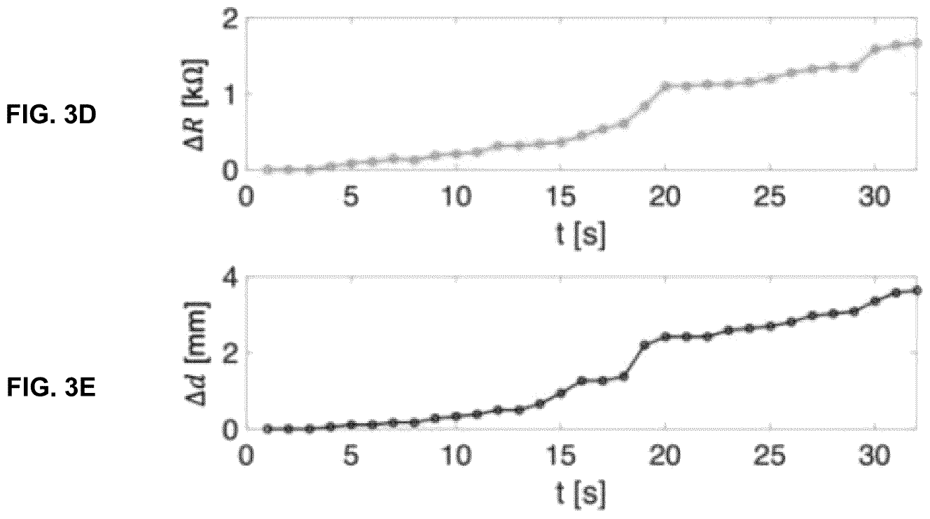

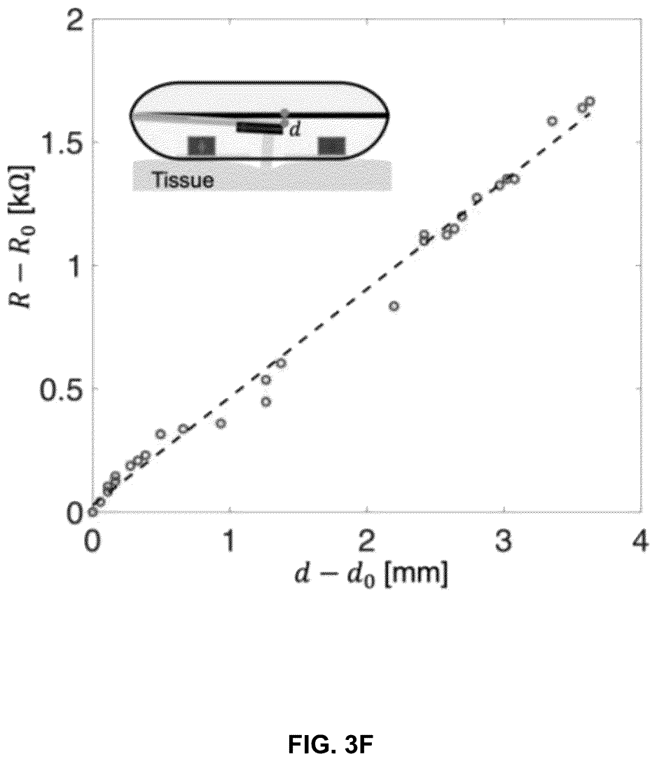

To illustrate the palpation mechanism, FIG. 3A presents a free-body diagram presenting the cantilever beam's interaction with both magnetic forces and torques. Through the application of an external magnetic field and its spatial gradient, the capsule device is drawn towards the soft material by magnetic pulling forces, denoted as Fg1 and Fg2, exerted on the two magnets affixed to its bottom shell. These pulling forces are generated by an external magnet (diameter: 25 mm, thickness: 20 mm, N45, NdFeB), strategically employed to produce the desired anchoring effect. Concurrently, the cantilever beam undergoes a bending motion, characterized by a bending displacement d, when the magnet positioned at its tip experiences a magnetic torque τ3 induced by the external magnetic field B along the z-axis. As the external magnetic field intensifies, the probe mounted on the cantilever beam's tip contacts the soft material, exerting pressure as the magnetic field reaches a sufficient strength. While the small magnet (5 mm by 3 mm by 1.5 mm, NdFeB) on the beam tip may also encounter a magnetic pulling force, the contribution of the pulling force to the beam deformation is significantly smaller due to the rapid decay of the external magnetic field gradient, highlighting the dominance of torque-induced deformation. Experimental images in FIG. 3B showcase two distinct states of a capsule device positioned on the surface of a bulk synthetic soft material (gel wax). When subjected to a magnetic field of 0.2 mT, the cantilever beam remains aloof from the soft material. However, upon increasing the magnetic field to 31.4 mT, sufficient deformation is observed in the cantilever beam, resulting in the compression of synthetic materials, as evidenced by the visualization of the black probe tip pressing against the surface.

In FIG. 3C, the magnitude of the external magnetic field was gradually increased from 0 mT to 31 mT. Correspondingly, the electrical resistance of the strain gauge sensor positioned on the top surface of the cantilever beam gradually ascends from 5.3 kΩ to 7.0 kΩ, as shown in FIG. 3D. This incremental rise mirrors the elongation experienced by the strain gauge sensor, serving as a direct indicator of its deformation. Furthermore, to comprehensively analyze the deformation of the cantilever beam, a video camera was used to track the position of its tip. The resulting data illustrates that the beam deflection d exhibits a parallel trend to the electrical resistance of the sensor, as presented in FIG. 3E. This correlation suggests that the electrical sensor resistance is a reliable estimator of palpation depth. In addition, FIG. 3F showcases a relatively linear mapping from the palpation depth to the change in electrical resistance. This observation highlights the potential utility of electric resistance calibration for accurately gauging the deformation of the capsule's cantilever beam, thus helping precise assessment of the palpation depth.

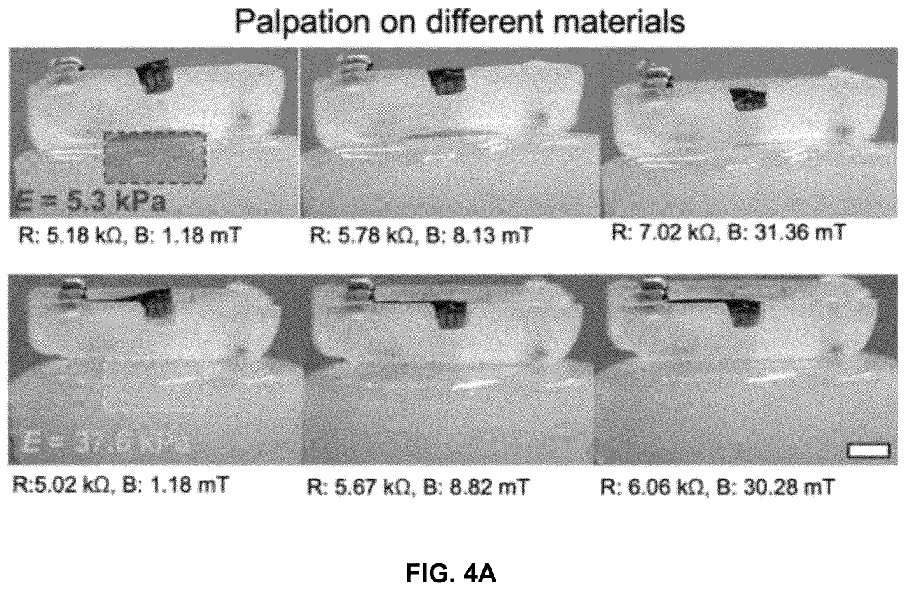

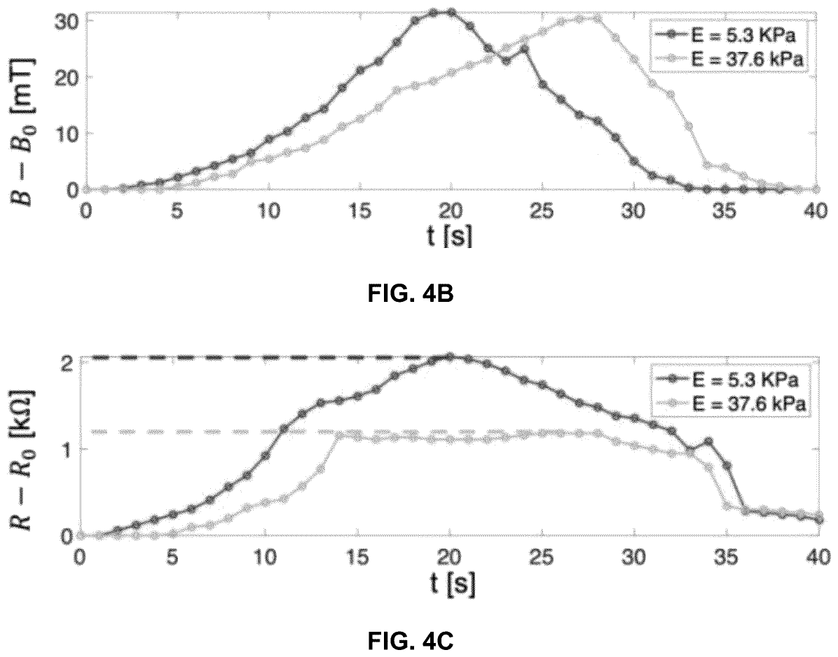

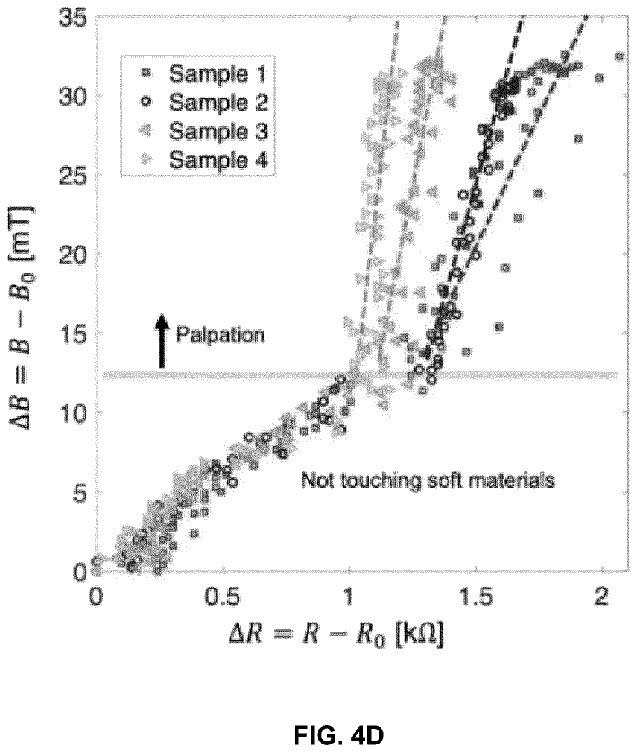

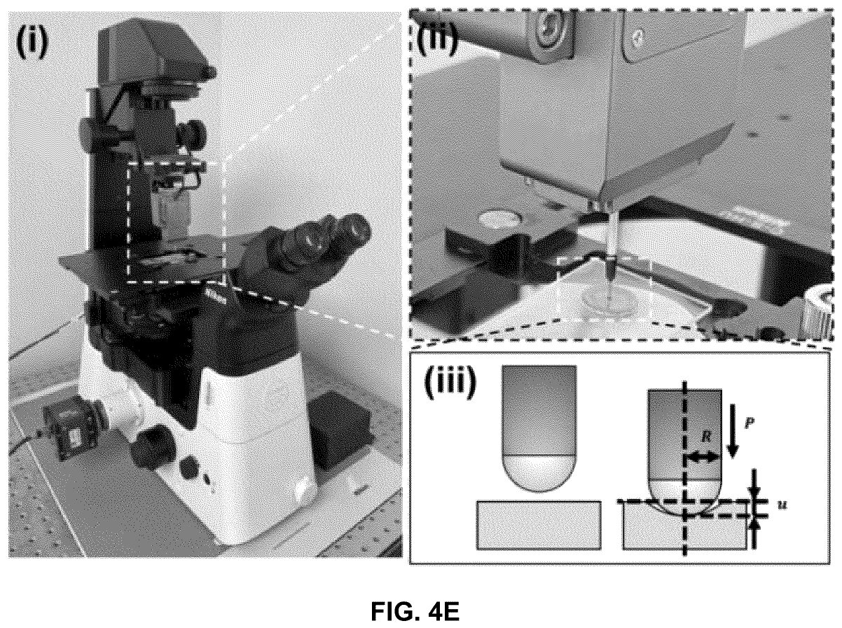

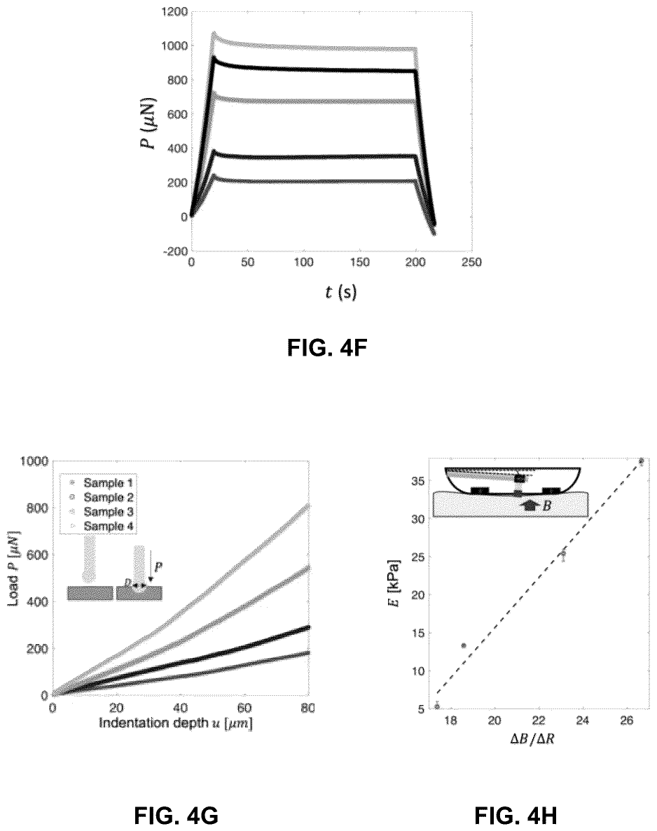

In the process of calibrating the capsule device for sensing material elasticity, the capsule to palpate materials with known Young's modulus was used, as shown in FIGS. 4A-4H. Soft material especially biological tissue is typically viscoelastic (C. F. Guimarães, L. Gasperini, A. P. Marques, R. L. Reis, Nature Reviews Materials 2020 5:5 2020, 5, 351) but here the focus is on the elasticity of the material by palpating the soft material relatively slowly (see Supplementary Note 1 “Modeling of soft tissues” for details). FIG. 4A illustrates the capsule's palpation of two synthetic soft materials exhibiting distinct behaviors under identical magnetic field conditions. Specifically, on the softer material (E=5.3 kPa), an electrical resistance of 7 kΩ is recorded at B=30 mT, while on the firmer material (E=37.6 kPa), the electrical resistance measures approximately 6 kΩ at B=31 mT. Throughout the palpation process, the time-varying electrical resistance (FIG. 4B) and magnetic field (FIG. 4C) are plotted.

In addition, FIG. 4D presents a plot of ΔB=B−B0 and ΔR=R−R0, revealing a relatively linear relationship. Notably, the slope angles of these curves differ, correlating with the tissue elasticity. The stress applied on the soft material by the capsule device is depending on the external magnetic field and the induced strain is related to the material deformation for a given probe. The linear relationship ΔB and ΔR is further explained in “Modeling of the palpation by the magnetic cantilever beam” of the “Experimental Section”. Lastly, to establish a mapping between the slope angle of the curve in FIG. 4D and the Young's modulus of the soft material, measurements of the tested materials were further conducted using a mechanical tester (Bruker Hysitron BioSoft Indenter, see Supplementary Note 2 “Load Relaxation and Hertzian models” for details). The indentation of the prepared synthetic soft materials is performed using a benchtop test tool—Hysitron BioSoft from Bruker (FIG. 4E). FIGS. 4F and 4G showcases the axial force and material deformation for five synthetic materials (see “Preparation of synthetic soft material” in the “Experimental Section” for the details of the synthetic materials) composed of different polymers, spanning Young's modulus from 5 kPa to 50 kPa. These comprehensive measurements contribute to refining an understanding of the relationship between electrical resistance changes and tissue elasticity, enabling more precise assessment and calibration of the capsule device's palpation capabilities (FIG. 4H). A focus is specifically on measuring the storage modulus of soft tissues, which serves as a critical biomarker in diseased tissues. To accurately sense the storage modulus, it is ensured that the palpation speed is deliberately slow compared to the material's characteristic time constant. During experiments, a controlled loading to the sensor probe on porcine colon tissues was applied and was held steady. As illustrated in FIGS. S5A-S5B of Exhibit 1 of U.S. Provisional Patent Application Ser. No. 63/723,903 (not shown), the palpation loading speed was intentionally slow relative to tissue dynamics, allowing tissue deformation to stabilize promptly upon initiating palpation.

### Example 3: Control Robot Motion by External Magnetic Fields

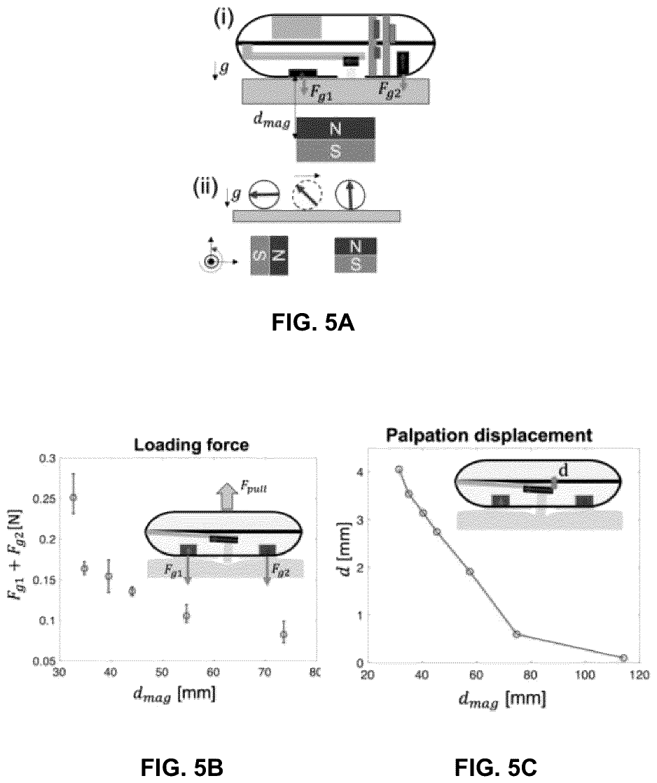

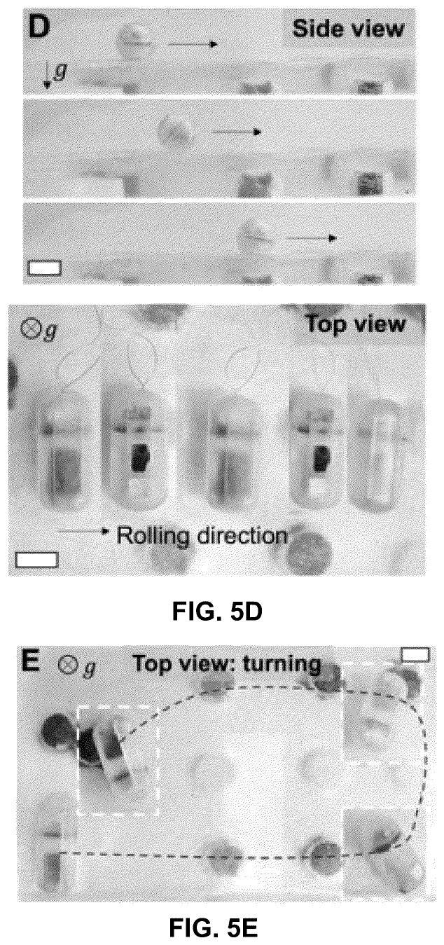

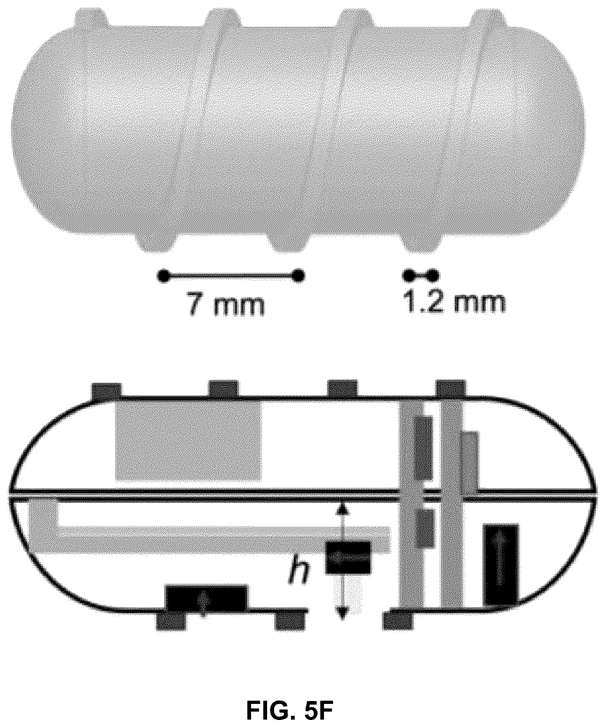

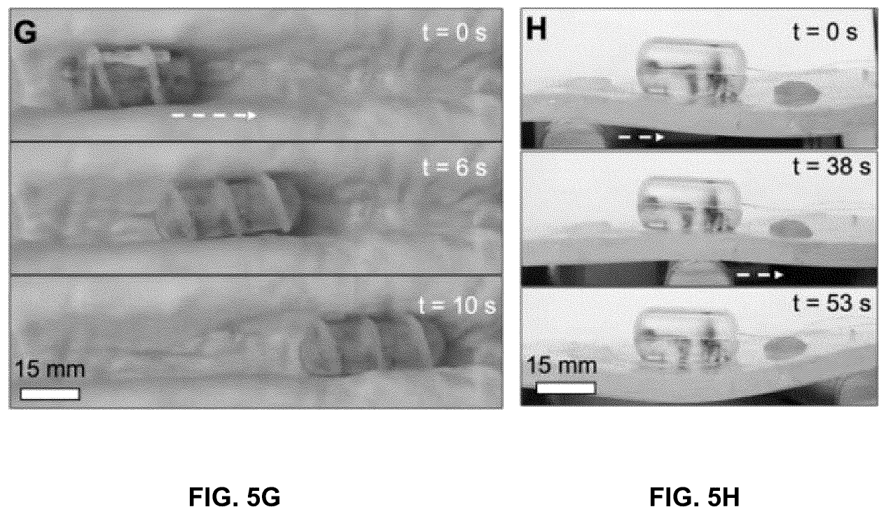

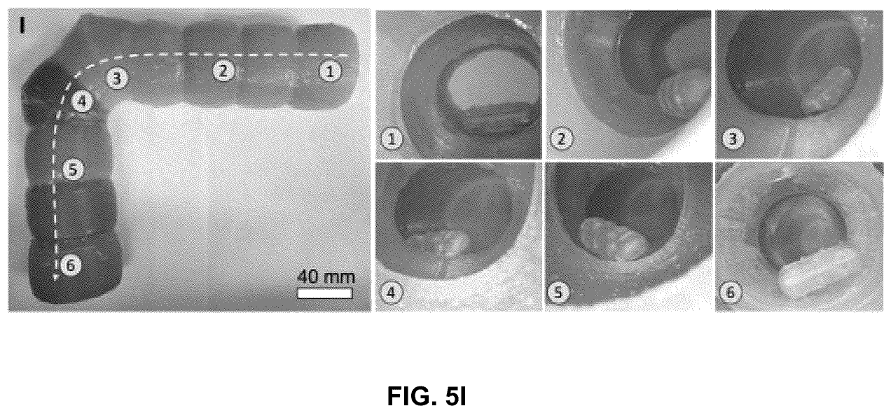

Ensuring the capsule device's secure anchoring on soft tissues is crucial for effective palpation. During the operation, it is important that the capsule device maintains sufficient pressure against the surface, facilitating robust interaction with the soft tissue via the palpation unit. This critical functionality is achieved through the magnetic gradient pulling force generated by the two permanent magnets affixed to the bottom cap of the capsule device, as illustrated in FIG. 5A. In scenarios where the magnetic field attains significant strength, an external magnet serves dual purposes: supplying the magnetic torque necessary to bend the cantilever beam and providing the essential magnetic gradient pulling force for pressing the capsule device against the tissue surface, as depicted in FIG. 5A(i). Conversely, when applying a rotating external magnetic field with a relatively modest magnitude, the capsule device seamlessly navigates across the material surface, effectively rolling to maneuver around obstacles, as illustrated in FIG. 5A(ii).

Considering that both the palpation-based sensing mechanism and robot locomotion are controlled by external magnetic fields, a quantitative analysis of how these two functions are controlled when varying the distance from the external magnet to the capsule device was performed. As shown in FIG. 5B, it was observed that the loading force is intricately linked to the intensity of the applied magnetic field. The loading force diminishes as the distance from the external magnet increases. Simultaneously, as illustrated in FIG. 5C, the palpation displacement on a given soft material is also decreasing when increasing the distance from the external magnet to the capsule. Therefore, to ensure precise control during experiments, the distance between the external magnet and the capsule device needs to be regulated utilizing a permanent magnet mounted on a motion stage. The actuation distance could be enlarged by using permanent magnets in a larger size as shown in FIGS. S6A-S6B of Exhibit 1 of U.S. Provisional Patent Application Ser. No. 63/723,903 (not shown). This distance control guarantees consistent and reliable magnetic field and its spatial gradient, enabling accurate evaluation of the capsule device's palpation capabilities across various scenarios and conditions.

Moreover, FIG. 5D provides a visualization of the rolling locomotion of the capsule device on a flat surface, initiated by rotation induced by an external magnetic field of a magnitude less than 10 mT. Both top and side views illustrate a capsule device being controlled to roll forward on a phantom. This movement is made possible through the combined action of the magnetic gradient pulling force and gravity, ensuring sufficient normal force to maintain friction and prevent slipping as the capsule device rolls. It should be noted that an advantage of the proposed capsule is its integrated on-board magnetic sensor, which allows for predicting the capsule body orientation in conjunction with the external magnetic field. This is crucial when the capsule's net magnetic moment is not fully aligned with the external magnetic field due to external distance. FIGS. S7A-S7G of Exhibit 1 of U.S. Provisional Patent Application Ser. No. 63/723,903 (not shown) illustrates the mechanism and validation of estimating the capsule's orientation by utilizing the sensed magnetic field in the local coordinate system of the on-board magnetic sensor, alongside the known external magnetic field in the global coordinate system. Additionally, FIGS. S8A-S8C of Exhibit 1 of U.S. Provisional Patent Application Ser. No. 63/723,903 (not shown) demonstrate that the capsule's location can be detected using an array of Hall-effect sensors, similar to a previous method (D. Son, X. Dong, M. Sitti, IEEE Transactions on Robotics 2019, 35, 343), in a proof-of-concept demonstration.

The steering capability of the capsule device in FIG. 5E was further demonstrated, achieved through its rolling motion on a gel wax phantom. Steering of the capsule is further accomplished by adjusting the plane of the rotating magnetic field using a 5-DOF magnetic actuation system (FIGS. S9A-S9B of Exhibit 1 of U.S. Provisional Patent Application Ser. No. 63/723,903 (not shown) and “Magnetic actuation system” in “Experimental Section”). This capability highlights the versatility and precision provided by the magnetic actuation system, potentially allowing the capsule device to navigate effectively within the GI tract for both tissue elasticity sensing and targeted movement. The integration of magnetic control mechanisms facilitates not only palpation but also controlled locomotion, crucial for navigating the intricate and often challenging terrain in the GI tract. Other locomotion such as walking (X. Dong, M. Sitti, Proc IEEE Int Conf Robot Autom 2017, 6612) and helical crawling (N. Shamsudhin, V. I. Zverev, H. Keller, S. Pane, P. W. Egolf, B. J. Nelson, A. M. Tishin, Med Phys 2017, 44, e91) can also be carried out by the capsule device such that a fine spatial palpation resolution can be realized. To navigate the capsule through folded tissues, a helical skin can be integrated onto the capsule body, allowing it to propel itself through friction, as shown in FIGS. 5F and 5G.

In addition, the peristaltic motion in the human GI tract may affect palpation by introducing disturbances. The anchoring force provides a pulling force to keep the capsule on the tissue surface. Since the sampling rate of the magnetic field and strain gauge is higher than that of the peristaltic motion, they can accurately capture data despite these movements. In FIG. 5H and FIGS. S10A-S10D of Exhibit 1 of U.S. Provisional Patent Application Ser. No. 63/723,903 (not shown), the sensing data was compared with and without peristaltic motion. The magnetic field and tissue deformation exhibit a similar mapping regardless of peristaltic motion (FIGS. S10A-S10D of Exhibit 1 of U.S. Provisional Patent Application Ser. No. 63/723,903 (not shown)). Lastly, the capsule device is demonstrated to navigate through a colon phantom with haustral structures, as shown in FIG. 5I, highlighting its potential for traversing luminal environments.

### Example 4: Demonstration of Palpating Heterogenous Material by the Capsule Device

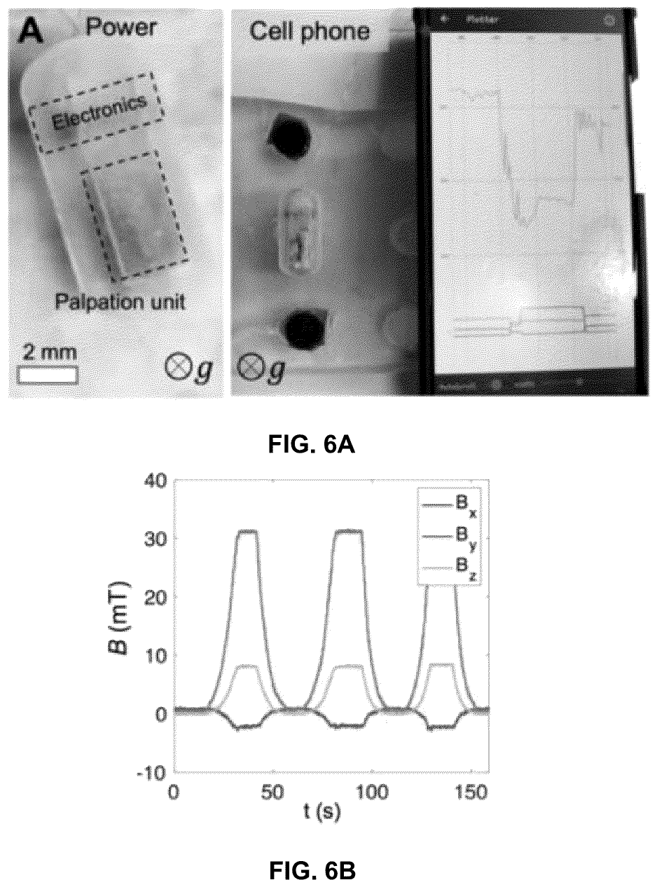

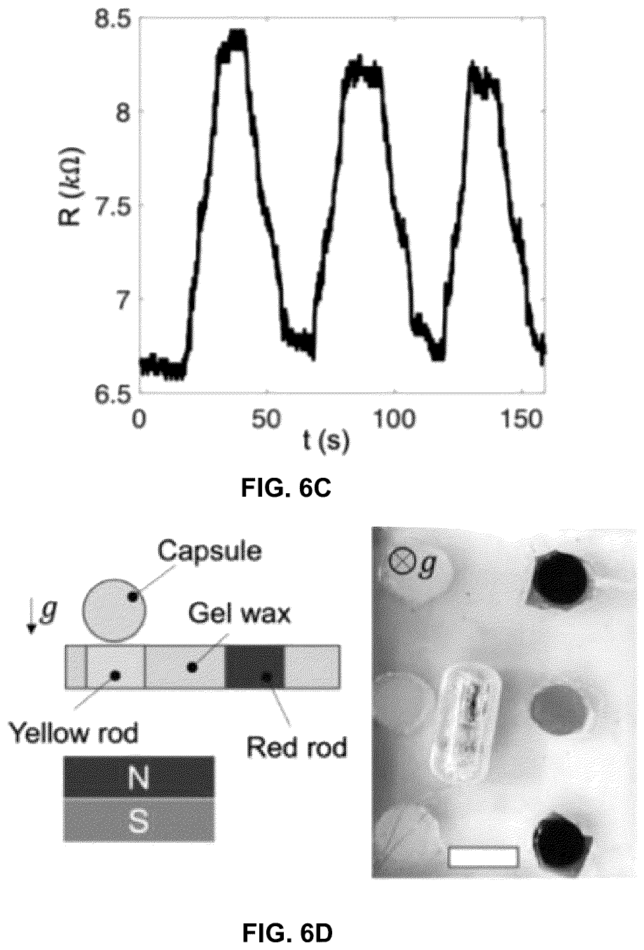

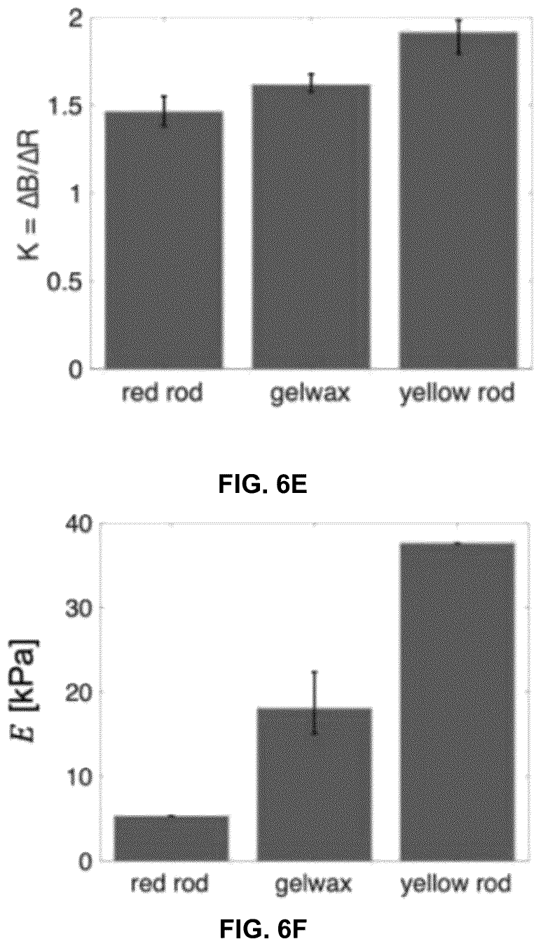

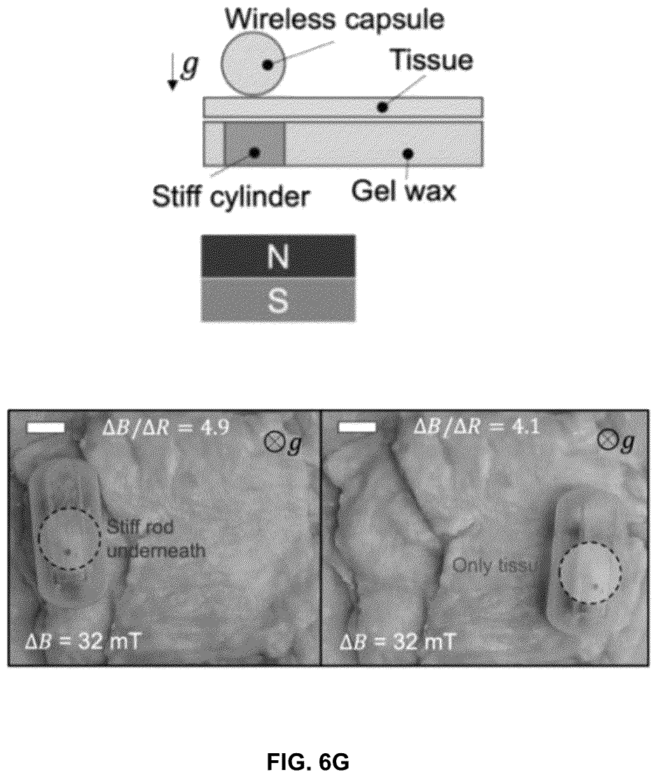

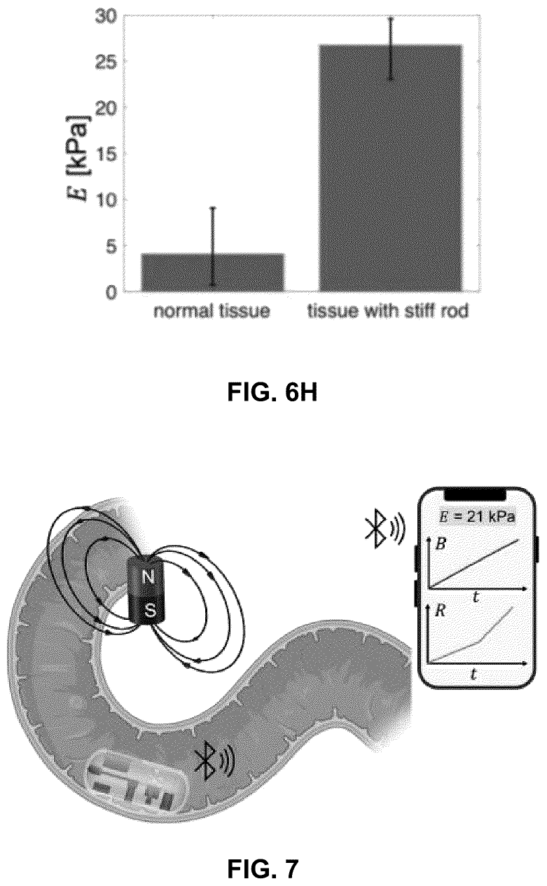

FIGS. 6A-6H illustrates the untethered locomotion and palpation capabilities of an assembled wireless capsule device, equipped with integrated sensors and communication units, requiring only two power wires for operation. In FIG. 6A, the wireless signal transmitted by the capsule device is seamlessly received by a cellphone and displayed via a dedicated app. Additionally, FIGS. 6B and 6C showcase the data pertaining to magnetic field and cantilever beam electrical resistance, respectively, received by a computer through BLE communication. These data sets are retrieved and plotted, providing valuable insights into the operational parameters of the capsule device.

Leveraging the capabilities of the capsule device, its proficiency in sensing material elasticity on a phantom characterized by heterogeneous material distribution was highlight. FIG. 6D captures the capsule device palpating the surface of the phantom, where polymer rods of varying elasticity are embedded. As the robot traverses the phantom surface, it palpates diverse materials and discerns their elasticity based on a meticulously calibrated model. The acquired signal and the estimated material elasticity are depicted in FIGS. 6E and 6F, respectively, showcasing the remarkable ability of the capsule device to spatially sense heterogeneous material properties. This capability holds significant promise for applications requiring precise assessment and characterization of tissue elasticity across varied compositions and distributions, underscoring the versatility and efficacy of the wireless capsule device platform.

To further underscore the potential application of the fully wirelessly controlled capsule device for sensing the elasticity of soft biological tissues, the same capsule device was employed to palpate porcine colon tissues ex vivo, as shown in FIG. 6G. Equipped with an integrated on-board battery, the capsule device operates fully wirelessly. As the robot actuation does not consume onboard energy, the on-board battery allows the wireless capsule device for a continuous operation duration of about 2.3 hours based on experimental tests (see “Bluetooth communication and power consumption” in the “Experimental Section”). Furthermore, palpation-based sensing was conducted using the capsule device on the tissue surface while utilizing a rigid cylinder (E=30 kPa) beneath the porcine colon tissue to simulate tumor tissues. Remarkably, the capsule device adeptly perceives the presence of the rigid cylinder beneath the tissue by comparing the strain gauge signal and the magnetic field signal with those when palpating the surrounding regular tissues, as illustrated in FIG. 6G. The material elasticities are further estimated using the calibrated model showing 20 kPa and 5 kPa for the abnormal and normal tissues, respectively, as shown in FIG. 6H. This capability highlights the ability of the capsule device to accurately distinguish areas of differing elasticity, crucial for identifying potential abnormalities such as fibrosis which typically show elasticity more than 15 kPa due to excessive extra cellular matrix deposition (L. A. Johnson, E. S. Rodansky, K. L. Sauder, J. C. Horowitz, J. D. Mih, D. J. Tschumperlin, P. D. Higgins, Inflamm Bowel Dis 2013, 19, 891). FIGS. S11A-S11F of Exhibit 1 of U.S. Provisional Patent Application Ser. No. 63/723,903 (not shown) also show the ability of the capsule palpating in a colon phantom with curved structures such as haustra structures. Therefore, the integration of active locomotion and on-demand palpation functionality makes the capsule device as a promising tool for sensing tissue elasticity, thus facilitating enhanced disease diagnosis. By offering real-time, wireless assessment capabilities, the proposed capsule device is promising for providing on-demand in situ diagnostic functions in various GI tract diseases.

### Example 5: Additional Discussion Relating to Examples 1-4

In summary, it has been disclosed herein a mechanism of sensing the elasticity of soft biological tissues by palpation enabled by remote magnetic actuation. The sensing mechanism is implemented on a capsule device with an on-board flexible magnetic actuator and wireless communication modules. The capsule device shows a unique functionality of active navigation and palpation of soft biological tissues, all remotely actuated and controlled by external magnetic fields. The navigation of the robot to a target area is achieved through a rolling locomotion, enabling precise and rapid position control in the GI tract for tissue examination. The magnetic gradient pulling force facilitates secure attachment of the capsule device to soft tissue surfaces, guaranteeing stable palpation. The captured tissue elasticity data is wirelessly transmitted via BLE signal, enabling seamless, real-time transfer for convenient access and analysis by medical professionals. The palpation-based sensing mechanisms have been validated by comparing with the ground truth measured by a high-precision mechanical indenter. Therefore, capsule devices described herein pave the way towards minimally invasive sensing of tissue elasticity, offering enhanced precision and efficiency in disease diagnostics.

The current design of the capsule device features a palpation depth limited to 2 to 3 mm, ideal for sensing in proximity to the intestinal tissue surfaces. To expand tissue sensing capabilities beneath the surface, a near-infrared light sensor (L. Zhang, C. D. Wallace, J. E. Erickson, C. M. Nelson, S. M. Gaudette, C. S. Pohl, S. D. Karsen, G. H. Simler, R. Peng, C. A. Stedman, et al., Scientific Reports 2020 10:1 2020, 10, 1; S. U. Bae, World J Gastroenterol 2022, 28, 1284) can be integrated, for example, for sensing the tissue deformation deeper under the intestine surface. It will enable monitoring of tissue deformation at deeper locations and provide texture information through LEDs emitting specific wavelengths (S. Y. Lee, J. M. Pakela, K. Na, J. Shi, B. J. McKenna, D. M. Simeone, E. Yoon, J. M. Scheiman, M. A. Mycek, Sci Adv 2020, 6, DOI). In addition, the current operational duration is constrained to about 2.3 hours, which is sufficient for mapping a small area. However, the operational time may be increased by optimizing the circuit to reduce power consumption or integrating an energy harvesting unit (J. C. Chen, P. Kan, Z. Yu, F. Alrashdan, R. Garcia, A. Singer, C. S. E. Lai, B. Avants, S. Crosby, Z. Li, et al., Nature Biomedical Engineering 2022 6:6 2022, 6, 706; P. Nadeau, D. El-Damak, D. Glettig, Y. L. Kong, S. Mo, C. Cleveland, L. Booth, N. Roxhed, R. Langer, A. P. Chandrakasan, et al., Nature Biomedical Engineering 2017 1:3). With these enhancements, prolonged operation time, enabling high-resolution palpation over extended periods can be realized. Moreover, the capsule device could be encapsulated by biocompatible material such as parylene-C (R. Correia, J. Deuermeier, M. R. Correia, J. Vaz Pinto, J. Coelho, E. Fortunato, R. Martins, ACS Appl Mater Interfaces 2022, 14, 46427) to ensure biocompatibility. Finally, the capsule could be integrated with additional modules for liquid biopsy (X. Dong, B. Xiao, H. Vu, H. Lin, M. Sitti, Sci Adv 2024, 10, 2758) or tissue biopsy (D. Son, H. Gilbert, M. Sitti, Soft Robot 2020, 7, 10), utilizing magnetically controlled pumps and valves (Y. Xu, H. Lin, B. Xiao, H. Tanoto, J. Berinstein, A. Khoshnaw, S. Young, Y. Zhou, X. Dong, Adv Healthc Mater 2024, 2402373; S. Sharma, L. C. Jung, N. Lee, Y. Wang, A. Kirk-Jadric, R. Naik, X. Dong, Adv Funct Mater 2024, 2405865.) to enable further sample analysis outside the body following palpation-based sensing.

Additional improvements can also be realized. First, the capsule needs to be sealed to prevent gastric liquid from leaking into the capsule body. Sealing with a Teflon film was demonstrated, showing that it does not affect palpation, as shown in FIGS. S12A-S12C of Exhibit 1 of U.S. Provisional Patent Application Ser. No. 63/723,903 (not shown). Additionally, the size of the capsule device can be reduced to avoid obstruction as capsule endoscopes of smaller dimensions have 1% chance of obstruction (C. M. Höög, L. Å. Bark, J. Arkani, J. Gorsetman, O. Broström, U. Sjöqvist, Gastroenterol Res Pract 2012, 2012, 518718). To reduce the risk of obstruction to the patient, as shown in FIGS. S13A-S13D of Exhibit 1 of U.S. Provisional Patent Application Ser. No. 63/723,903 (not shown), the size of the demonstrated capsule, originally about 15 mm in diameter and 32 mm in length, has been reduced to 11.6 mm in diameter by 32 mm in length. Following the specifications of the FDA-approved PillCam Colon capsule (L. Negreanu, R. Babiuc, A. Bengus, R. Sadagurschi, World J Gastrointest Endosc 2013, 5, 559) from Medtronic Inc, this size scaling-down reduces the risk of a capsule device for GI tract obstruction. In addition, integrating a camera into palpation capsules described herein may further allow identifying suspicious tissue areas. One alternative solution may be integrating a small magnet onto a commercial endoscope which could be further used to monitor a palpation capsule (see FIGS. S14A-S14B of Exhibit 1 of U.S. Provisional Patent Application Ser. No. 63/723,903 (not shown) for proof-of-concept) to facilitate tissue identification.

The palpation-based sensing mechanism described herein and the implemented capsule device described herein offer a minimally invasive and non-disruptive method for evaluating tissue elasticity, making it a promising technology for disease diagnosis, treatment assessment, and early detection, especially in conditions like intestine fibrosis and early-stage colorectal cancer. With its innovative features including active steering, tissue palpation, wireless data transmission, and power supply, capsule devices described herein hold the potential to advance medical practices and improve patient outcomes in diagnosis and monitoring IBD, colorectal cancer, and other diseases.

### Example 6: Experimental Details Relating to Examples 1-4

### Preparing the Graphene Layer:

A three-step laser-based approach was utilized to fabricate all components of the LIG-based sensor (J. Han, X. Dong, Z. Yin, S. Zhang, M. Li, Z. Zheng, M. C. Ugurlu, W. Jiang, H. Liu, M. Sitti, Proc Natl Acad Sci USA 2023, 120, e2308301120). To initiate the LIG layer generation, a single-side polyimide tape (63 μm, Kapton Tape) was adhered to a supporting glass substrate employing a sacrificial Polyvinyl Alcohol (PVA) layer (˜10 μm thickness, PVA, Sigma-Aldrich) within a 60-W CO₂ laser cutter (wavelength: 10.6 μm, beam size: ˜120 μm, VLS3.60DT, Universal Laser Systems). The engraving mode with laser power (30%), laser speed (50%) and laser points-perinch (PPI) (1,000) were used to generate the graphene. Uncured PDMS was applied onto the engraved PI tape and cured at 110° C. for 20 mins to transfer the LIG onto the PDMS layer. The thickness of the PDMS layer was adjusted to approximately 120 μm using spacer tapes (63 μm per layer for two layers). Subsequently, the top surface of the uncured PDMS was scraped using a single-edge razor blade. Finally, due to the weak bonding between the sacrificial layer and the glass substrate, the PI tape was easily peeled off after curing. The PDMS layer, along with the LIG layer, was peeled off from the PI tape due to the strong bonding between the PDMS and LIG layers, facilitated by uncured PDMS infiltrating into the porous LIG structure.

### Patterning the LIG Strain Gauge Sensor:

The strain gauge was patterned utilizing a UV laser system (LPKF ProtoLaser U4, LPKF Laser & Electronics AG) which has key parameters including wavelength of 355 nm, beam size of 15 μm, and accuracy of 2 μm. An engraving method was used for precisely patterning the PDMS-LIG sheets with a line width of approximately 20 μm. Selective sintering and removal of the graphene layer were achieved using a low-power laser beam with a power of 2.3 W and a speed of 600 mm/s in the engraving mode, ensuring both sensor sensitivity and stability. Subsequently, a high-power laser beam with a power of 3.5 W, and a speed of 350 mm/s in the cutting mode was utilized to cut the strain gauge sensor from the PDMS-LIG sheet, adhering to an example dimension of 10 mm by 6 mm by 0.2 mm (length by width by thickness) in FIGS. 2A-2E and a different design embodiment in FIG. S15 (see, Exhibit 1 of U.S. Provisional Patent Application Ser. No. 63/723,903 (not shown)).

### Bluetooth Low Energy Communication and Power Consumption:

The customized PCB boards for the on-board electronic components were designed using an online electronic design automation (EDA) software (EasyEDA) and fabricated with a UV laser system (LPKF ProtoLaser U4, LPKF Laser & Electronics AG). The designs of the circuit boards were described in FIGS. S2A and S2B of Exhibit 1 of U.S. Provisional Patent Application Ser. No. 63/723,903 (not shown) and FIGS. S3A and S3B of Exhibit 1 of U.S. Provisional Patent Application Ser. No. 63/723,903 (not shown). The electronic components of the capsule device comprise a wireless communication module and a battery module. The wireless communication module features an nRF52832 Bluetooth LE SoC (MDBT42V-512KV2 and MDBT42V-P512KV2, Raytac Corporation). Connected to this was a 4.5 V, 18 mAh (3LR626, Enerpe) battery, a three-axis magnetic sensor (TLV493D-A1B6, Infineon AG), and other typical electrical components necessary to support both modules. The printed circuit board (PCB) utilized is an FR4 (0.85 mm thick), plated with 0.35 μm of copper, and fabricated using a UV laser machine (LPKF Protolaser U4). Programming of the nRF52832 chip was done within the Arduino Integrated Development Environment. The compiled hex file was uploaded using the Nordic nRFConnect Programmer via the Serial Wire Debug (SWD) protocol. The nRF52832 chip communicates with the three-axis magnetic sensor through I2C to collect magnetic field data at approximately 1 Hz. It also read the voltage at its analog pin connected to the middle node of a simple voltage divider for the strain gauge sensor, comprising a 6.8 kΩ resistor and the strain gauge sensor, every 0.2 milliseconds for rapid sensing. This collected information was promptly transmitted to a personal computer or cell phone via Bluetooth LE signal. The Bluetooth LE signal can be interpreted using various programs such as the Adafruit Bluefruit App or a customized script in Matlab 2022b (MathWorks). During the operation, the system was measured to draw a current of approximately 2.41 mA. For prolonged usage, the battery stabilized under the load of the nRF52832 to 3.7 V, resulting in a power consumption of 8.92 mW. With a coin Alkaline battery of 18 mAh, 4.5V (Type: 3LR626, Enerpe), the device remained active for 2 hours and 20 minutes.

### Modeling of the Palpation by the Magnetic Cantilever Beam:

When a rigid cylinder is pressed onto an elastic material such as biological tissues, the material palpation force could be estimated by using a cantilever beam model. For the cantilever beam shown in FIG. 3A, the moment balancing equation is had:

$${N}_{3} {L}_{c} = {m}_{3} B sin θ - {K}_{c} d \qquad ( 1 )$$

where d=Lc sin θ, m3 is the magnetic moment of the magnet on the tip of the cantilever beam, and Kc is the spring coefficient of the cantilever beam. When the probe starts touching the soft material, sin θ is relatively constant (θ is close to π/2), as the deflection of the cantilever beam d is relatively small. Meanwhile, based on the Hertzian contact theory (C. E. Wu, K. H. Lin, J. Y. Juang, Tribol Int 2016, 97, 71; I. N. Sneddon, Int J Eng Sci 1965, 3, 47), the reaction force applied to the soft material by the probe N3′ equals to N3, given by:

$${{N}_{3}}^{′} = 2 {R}_{p} {E}^{*} d \qquad ( 2 )$$

where Rp is the radius of the cylindrical probe and E*=E/(1−v²), E is the Young's modulus of the elastic material, and v is the Poisson ratio. With Equations (1) and (2), it is had:

$$B = \frac{1}{{m}_{3} sin θ} [ \frac{2 {L}_{c} {R}_{p} E}{1 - {v}^{2}} + {K}_{c} ] d \qquad ( 3 )$$

### Magnetic Actuation System:

A mobile permanent magnet system (see, FIGS. S7A-S7G of Exhibit 1 of U.S. Provisional Patent Application Ser. No. 63/723,903 (not shown)) was used for actuating the capsule device to navigate. The mobile permanent magnet used was an NdFeB magnet with a dimension of 25 mm by 25 mm by 25 mm, which generated a magnetic field of up to 50 mT at a maximum frequency of 5 Hz, offering versatile and dynamic magnetic actuation capabilities. The permanent magnet was mounted on a 2-DOF rotational motion stage which was further mounted to a 3-DOF translational motion stage, all actuated using a dual H-Bridge motor driver (L298N). The magnetic actuation system with 5-DOF magnetic field control was teleoperated by a joystick.

### Preparation of Synthetic Soft Material and Porcine Tissues:

A series of soft materials was prepared to test and validate the capsule device's sensing mechanism. The mixture involved Ecoflex 00-30 (Smooth-On, Inc.) blended with a polymer slacker (Smooth-On, Inc.) in various proportions, aimed at achieving soft materials with specific Young's modulus values. To ensure optimal quality, the uncured polymer underwent a degassing procedure inside a vacuum chamber, effectively eliminating any excess gas during the mixing process. For shaping, cylindrical molds measuring 15 mm in diameter by 20 mm in height were prepared using 3D printing. Subsequently, the degassed polymer mixtures were poured into these molds and carefully cured on a hot plate set at 80° C. Upon solidification, the polymer samples were delicately extracted from the molds for further testing. In addition, fresh porcine colon tissues were purchased from a local slaughterhouse, Tennessee, USA. The tissue was kept frozen in a freezer and defrosted before experiments. The porcine tissue was cut and attached to a phantom substrate made of gel wax with embedded cylinders made of materials of different elastic modulus.

### Example 7: Additional Details

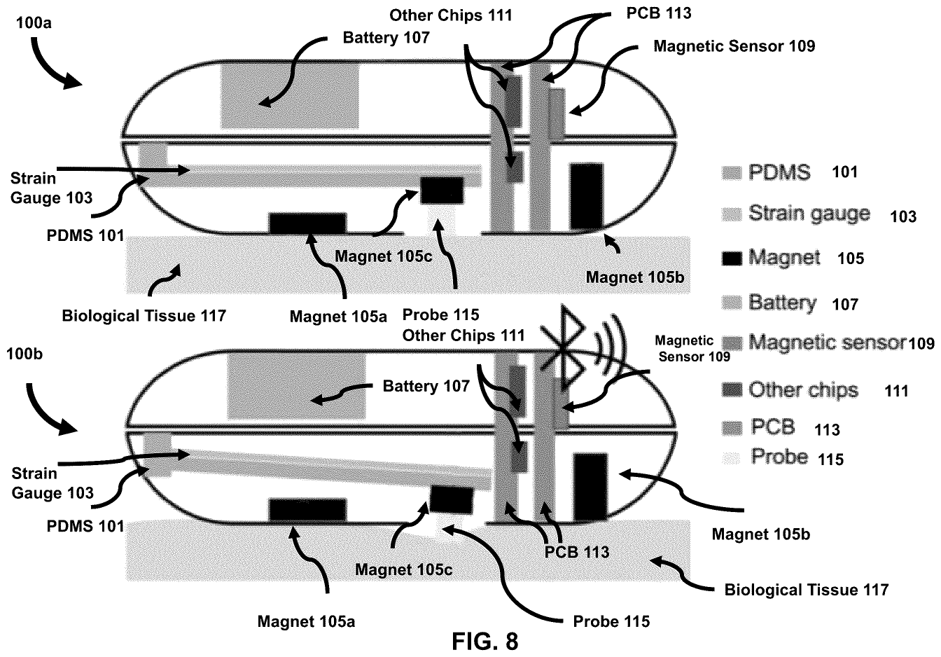

FIGS. S1A-S1B of U.S. Provisional Patent Application Ser. No. 63/723,903 show an embodiment of Capsule dimensions and Experimental Images. FIG. 8A. Dimensions of the capsule robot caps. FIG. 8B. Optical image of the electronics components inside the capsule robot. Scale bars, 6 mm.

### Example 8: Additional Details

FIG. 8 shows additional aspects of the present disclosure, in particular an embodiment of a capsule device in a relaxed 100a position and palpitation unit in a deflected position 100b with the probe 115 touching the biological tissue 117. As can be seen in FIG. 8, the palpitation unit (comprising strain gauge 103 and PDMS 111) is one position in 100a and a sensor position in 100b where it is deflected. PDMS 101 and the strain gauge 103 are shown.

Magnets 105a and 105b aid in translocation of the capsule device in certain aspects and magnet 105c of the palpitation unit aids in sensing. A power source is shown with the battery 107, as well as the magnetic sensor 109, other chips 111 (Bluetooth, or other wireless communication, for example), PCB 113 and probe 115.

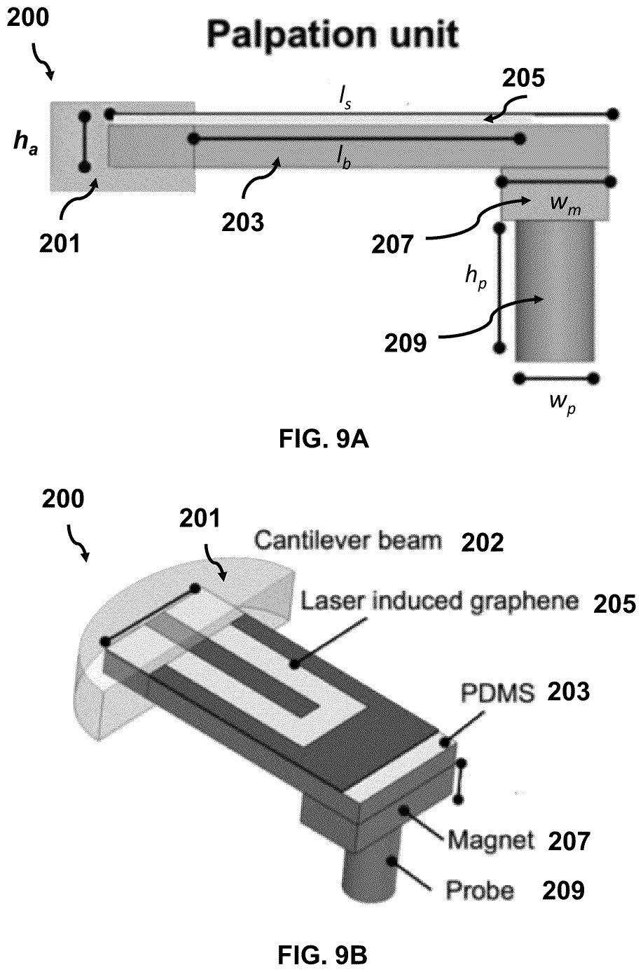

FIGS. 9A and 9B show additional aspects of an embodiment of a palpitation unit 200 according to the present disclosure. FIG. 9A shows a sideview whereas FIG. 9B shows a perspective view. The palpitation unit can comprise an anchor 201 that the cantilever beam 202 is fixed on or otherwise into on a side opposite the magnet 207 and probe 209. The cantilever beam comprises a strain gauge (for example the laser-induced graphene 205) that is disposed upon a plastic layer (for example, the PDMS 203). The magnet and the probe can flex up or down relative to the holder. As denoted in FIG. 9A, the anchor 201 can have a height (hₐ), whereas the strain gauge 205 and backing layer (PDMS 203) can have a length Iₛ and I_{b}, respectively, the magnet 207 and probe 209 can both have a width, wₘ and wₚ, respectively, and the probe can have a height hₚ.

### REFERENCES RELATED TO THE PRESENT DISCLOSURE

[1] S. Mascharak, J. L. Guo, M. Griffin, C. E. Berry, D. C. Wan, M. T. Longaker, Nature Reviews Bioengineering 2024 2:4 2024, 2, 305.
[2] L. A. Johnson, E. S. Rodansky, K. L. Sauder, J. C. Horowitz, J. D. Mih, D. J. Tschumperlin, P. D. Higgins, Inflamm Bowel Dis 2013, 19, 891.
[3] J. Zhao, D. Liao, R. Wilkens, K. Krogh, H. Glerup, H. Gregersen, Acta Biomater 2021, 130, 332.
[4] S. D'Alessio, F. Ungaro, D. Noviello, S. Lovisa, L. Peyrin-Biroulet, S. Danese, Nature Reviews Gastroenterology & Hepatology 2021 19:3 2021, 19, 169.
[5] F. Baidoun, K. Elshiwy, Y. Elkeraie, Z. Merjaneh, G. Khoudari, M. T. Sarmini, M. Gad, M. Al-Husseini, A. Saad, Curr Drug Targets 2020, 22, 998.
[6] C. F. Guimarães, L. Gasperini, A. P. Marques, R. L. Reis, Nature Reviews Materials 2020 5:5 2020, 5, 351.
[7] M. Jang, J. An, S. W. Oh, J. Y. Lim, J. Kim, J. K. Choi, J. H. Cheong, P. Kim, Nature Biomedical Engineering 2020 5:1 2020, 5, 114.
[8] J. Bauer, M. A. B. Emon, J. J. Staudacher, A. L. Thomas, J. Zessner-Spitzenberg, G. Mancinelli, N. Krett, M. T. Saif, B. Jung, Scientific Reports 2020 10:1 2020, 10, 1.
[9] K. Nan, V. R. Feig, B. Ying, J. G. Howarth, Z. Kang, Y. Yang, G. Traverso, Nature Reviews Materials 2022 7:11 2022, 7, 908.
[10] M. Beccani, C. Di Natali, C. E. Benjamin, C. S. Bell, N. E. Hall, P. Valdastri, Sens Actuators A Phys 2015, 223, 180.
[11] S. Mckinley, A. Garg, S. Sen, R. Kapadia, A. Murali, K. Nichols, S. Lim, S. Patil, P. Abbeel, A. M. Okamura, et al., IEEE International Conference on Automation Science and Engineering 2015, 2015-October 1151.
[12] I. Sack, Nature Reviews Physics 2022 5:1 2022, 5, 25.
[13] F. Avila, B. Caron, G. Hossu, K. Ambarki, S. Kannengiesser, F. Odille, J. Felblinger, S. Danese, M. Choukour, V. Laurent, et al., Dig Dis Sci 2022, 67, 4518.
[14] E. Song, Z. Xie, W. Bai, H. Luan, B. Ji, X. Ning, Y. Xia, J. M. Baek, Y. Lee, R. Avila, et al., Nature Biomedical Engineering 2021 5:7 2021, 5, 759.
[15] X. Yu, H. Wang, X. Ning, R. Sun, H. Albadawi, M. Salomao, A. C. Silva, Y. Yu, L. Tian, A. Koh, et al., Nature Biomedical Engineering 2018 2:3 2018, 2, 165.
[16] M. Lin, H. Hu, S. Zhou, S. Xu, Nature Reviews Materials 2022 7:11 2022, 7, 850.
[17] B. J. Nelson, S. Gervasoni, P. W. Y. Chiu, L. Zhang, A. Zemmar, Proceedings of the IEEE 2022, 110, 1028.
[18] B. Xiao, Y. Xu, S. Edwards, L. Balakumar, X. Dong, Adv Funct Mater 2024, 34, 2307751.
[19] C. Wang, Y. Wu, X. Dong, M. Armacki, M. Sitti, Sci Adv 2023, 9, DOI 10.1126/sciadv.adg3988.
[20] N. Shamsudhin, V. I. Zverev, H. Keller, S. Pane, P. W. Egolf, B. J. Nelson, A. M. Tishin, Med Phys 2017, 44, e91.
[21] J. Min, Y. Yang, Z. Wu, W. Gao, Adv Ther (Weinh) 2020, 3, 1900125.
[22] G. Ciuti, R. Caliò, D. Camboni, L. Neri, F. Bianchi, A. Arezzo, A. Koulaouzidis, S. Schostek, D. Stoyanov, C. M. Oddo, et al., Journal of Micro-Bio Robotics 2016 11:1 2016, 11, 1.
[23] G. Traverso, V. Finomore, J. Mahoney, J. Kupec, R. Stansbury, D. Bacher, B. Pless, S. Schuetz, A. Hayward, R. Langer, et al., Device 2023, 1, 100125.
[24] M. Mimee, P. Nadeau, A. Hayward, S. Carim, S. Flanagan, L. Jerger, J. Collins, S. McDonnell, R. Swartwout, R. J. Citorik, et al., Science (1979) 2018, 360, 915.
[25] J. M. Stine, K. L. Ruland, L. A. Beardslee, J. A. Levy, H. Abianeh, S. Botasini, P. J. Pasricha, R. Ghodssi, Adv Healthc Mater 2023, 2302897.
[26] C. Steiger, A. Abramson, P. Nadeau, A. P. Chandrakasan, R. Langer, G. Traverso, Nature Reviews Materials 2018 4:2 2018, 4, 83.
[27] C. E. Wu, K. H. Lin, J. Y. Juang, Tribol Int 2016, 97, 71.
[28] D. Son, X. Dong, M. Sitti, IEEE Transactions on Robotics 2019, 35, 343.
[29] X. Dong, M. Sitti, Proc IEEE Int Conf Robot Autom 2017, 6612.
[30] L. Zhang, C. D. Wallace, J. E. Erickson, C. M. Nelson, S. M. Gaudette, C. S. Pohl, S. D. Karsen, G. H. Simler, R. Peng, C. A. Stedman, et al., Scientific Reports 2020 10:1 2020, 10, 1.
[31] S. U. Bae, World J Gastroenterol 2022, 28, 1284.
[32] S. Y. Lee, J. M. Pakela, K. Na, J. Shi, B. J. McKenna, D. M. Simeone, E. Yoon, J. M. Scheiman, M. A. Mycek, Sci Adv 2020, 6, DOI 10.1126/SCIADV.ABC1746/SUPPL_FILE/ABC1746_SM.PDF.
[33] J. C. Chen, P. Kan, Z. Yu, F. Alrashdan, R. Garcia, A. Singer, C. S. E. Lai, B. Avants, S. Crosby, Z. Li, et al., Nature Biomedical Engineering 2022 6:6 2022, 6, 706.
[34] P. Nadeau, D. El-Damak, D. Glettig, Y. L. Kong, S. Mo, C. Cleveland, L. Booth, N. Roxhed, R. Langer, A. P. Chandrakasan, et al., Nature Biomedical Engineering 2017 1:3 2017, 1, 1.
[35] R. Correia, J. Deuermeier, M. R. Correia, J. Vaz Pinto, J. Coelho, E. Fortunato, R. Martins, ACS Appl Mater Interfaces 2022, 14, 46427.
[36] X. Dong, B. Xiao, H. Vu, H. Lin, M. Sitti, Sci Adv 2024, 10, 2758.
[37] D. Son, H. Gilbert, M. Sitti, Soft Robot 2020, 7, 10.
[38] Y. Xu, H. Lin, B. Xiao, H. Tanoto, J. Berinstein, A. Khoshnaw, S. Young, Y. Zhou, X. Dong, Adv Healthc Mater 2024, 2402373.
[39] S. Sharma, L. C. Jung, N. Lee, Y. Wang, A. Kirk-Jadric, R. Naik, X. Dong, Adv Funct Mater 2024, 2405865.
[40] C. M. Höög, L. Å. Bark, J. Arkani, J. Gorsetman, O. Broström, U. Sjöqvist, Gastroenterol Res Pract 2012, 2012, 518718.
[41] L. Negreanu, R. Babiuc, A. Bengus, R. Sadagurschi, World J Gastrointest Endosc 2013, 5, 559.
[42] J. Han, X. Dong, Z. Yin, S. Zhang, M. Li, Z. Zheng, M. C. Ugurlu, W. Jiang, H. Liu, M. Sitti, Proc Natl Acad Sci USA 2023, 120, e2308301120.
[43] I. N. Sneddon, Int J Eng Sci 1965, 3, 47.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A palpitation unit, comprising:
a double-layer cantilever beam comprising a strain gauge sensor and a back layer, wherein the double-layer cantilever beam is physically deformable along at least one axis;
a magnet; and
a palpitation probe.

2. The palpitation unit of claim 1, wherein the back layer of the double-layer cantilever beam comprises polydimethylsiloxane (PDMS).

3. The palpitation unit of claim 1, wherein the strain gauge sensor comprises laser-induced graphene (LIG).

4. The palpitation unit of claim 1, wherein the palpitation probe comprises a cylindrical structure.

5. The palpitation unit of claim 1, wherein the palpitation probe is on a side of the magnet opposite the back layer.

6. The palpitation unit of claim 1, wherein the magnet and the palpitation probe are on a distal end of the palpitation unit along the deformation axis.

7. A capsule device, comprising:
the palpitation unit of claim 1;
a power source;
a magnetic sensor; and
a wireless communication transceiver.

8. The capsule of claim 7, wherein the magnet of the palpitation probe is situated in between a first and a second magnet of the magnetic sensor.

9. The capsule of claim 7, wherein the wireless communication transceiver comprises a Bluetooth Low Energy (BLE) System-on-a-Chip (SoC).

10. A system, comprising:
a capsule device of claim 7;
an external magnetic field generator; and
a mobile application in wireless communication with the capsule device, the external magnetic field generator, or both.

11. The system of claim 10, wherein the external magnetic field generator comprises a one or more magnets.

12. The system of claim 10, further comprising a personal computing device comprising the mobile application.

13. A method of using a system, comprising:
administering a capsule of claim 7 to a subject in need thereof;
administering a magnetic field to the subject with a magnetic field generator;
collecting measurements from the capsule in the presence of the administered magnetic field with a mobile application.

14. The method of claim 13, wherein the subject in need thereof is a subject having or suspected of having Chrohn's disease or inflammatory bowel disease.

15. The method of claim 13, wherein the capsule is administered orally to the subject.

16. The method of claim 13, wherein the magnetic field is administered to a region of interest (ROI) of the subject.

17. The method of claim 13, wherein the ROI is a muscosa-interfacing region of the subject.

18. A method of making a deformable strain gauge sensor, comprising:
inducing a layer of graphene on a planar substrate;
casting the graphene layer with a liquid polymer;
transferring the casted layer onto a cured back layer; and
patterning the transferred layer.

19. The method of claim 18, wherein the inducing is with a carbon dioxide (CO₂) laser.

20. The method of claim 18, wherein the patterning is facilitated by applying negative pressure simultaneously in the delamination process of a conductor on a printed circuit board (PCB).
